SEQUENCE LISTING (28 sequences)

SEQ ID NO: 1 - protein, 321 aa, Escherichia coli
MKDNTVPLKLIALLANGEFHSGEQLGETLGMSRAAINKHIQTLRDWGVDVFTVPGKGYSLPEPIQLLNAKQILGQLDGGSVAVLPVIDSTNQYLLDRIGELKSGDACIAEYQQAGRGRRGRKWFSPFGANLYLSMFWRLEQGPAAAIGLSLVIGIVMAEVLRKLGADKVRVKWPNDLYLQDRKLAGILVELTGKTGDAAQIVIGAGINMAMRRVEESVVNQGWITLQEAGINLDRNTLAAMLIRELRAALELFEQEGLAPYLSRWEKLDNFINRPVKLIIGDKEIFGISRGIDKQGALLLEQDGIIKPWMGGEISLRSAEK

SEQ ID NO: 2 - protein, 258 aa, synthetic construct
MIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEK

SEQ ID NO: 3 - protein, 343 aa, Escherichia coli
MTNFYKVCLAVFILVCCNISHAAVSFIGSTENDVGPSQGSYSSTHAMDNLPFVYNTGYNIGYQNANVWRISGGFCVGLDGKVDLPVVGSLDGQSIYGLTEEVGLLIWMGDTNYSRGTAMSGNSWENVFSGWCVGNYVSTQGLSVHVRPVILKRNSSAQYSVQKTSIGSIRMRPYNGSSAGSVQTTVNFSLNPFTLNDTVTSCRLLTPSAVNVSLAAISAGQLPSSGDEVVAGTTSLKLQCDAGVTVWATLTDATTPSNRSDILTLTGASTATGVGLRIYKNTDSTPLKFGPDSPVKGNENQWQLSTGTETSPSVRLYVKYVNTGEGINPGTVNGISTFTFSYQ

SEQ ID NO: 4 - protein, 157 aa, synthetic construct
MAVSFIGSTENDVGPSQGSYSSTHAMDNLPFVYNTGYNIGYQNANVWRISGGFCVGLDGKVDLPVVGSLDGQSIYGLTEEVGLLIWMGDTNYSRGTAMSGNSWENVFSGWCVGNYVSTQGLSVHVRPVILKRNSSAQYSVQKTSIGSIRMRPYNGSS

SEQ ID NO: 5 - protein, 47 aa, synthetic construct
MLGFVGRVAAAPASGALRRLTPSASLPPAQLLLRAAPTAVHPVRDYA

SEQ ID NO: 6 - protein, 9 aa, synthetic construct
MGCINSKRK

SEQ ID NO: 7 - protein, 7 aa, synthetic construct
PKKKRKV

SEQ ID NO: 8 - protein, 9 aa, synthetic construct
LPPLERLTL

SEQ ID NO: 9 - protein, 14 aa, synthetic construct
GKPIPNPLLGLDST

SEQ ID NO: 10 - protein, 9 aa, synthetic construct
YPYDVPDYA

SEQ ID NO: 11 - protein, 257 aa, synthetic construct
IPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEK

SEQ ID NO: 12 - DNA, 471 bp, synthetic construct
  source 1..471: mol_type = other DNA
atggccgtgtccttcatcggcagcaccgaaaatgatgtgggccctagccagggcagctacagctctacacacgccatggacaacctgcctttcgtgtacaacaccggctacaatatcggctaccagaacgccaacgtgtggcggatctctggcggcttttgtgttggcctggacggcaaagtggatctgcctgttgtgggctctctggacggccagtctatctacggcctgacagaggaagtgggcctgctgatctggatgggcgacaccaattacagcagaggcacagccatgagcggcaacagctgggagaatgtgttcagcggatggtgcgtgggcaactacgtgtccacacagggactgtctgtgcacgtgcggcctgtgatcctgaagagaaatagcagcgcccagtacagcgtgcagaaaaccagcatcggctccatcagaatgcggccctacaatggcagctct

SEQ ID NO: 13 - DNA, 771 bp, synthetic construct
  source 1..771: mol_type = other DNA
atcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctatgctgatccgagagctgcgggcagccctggaactgttcgagcaggaaggcctggctccatatctgtcacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaag

SEQ ID NO: 14 - DNA, 72 bp, synthetic construct
  source 1..72: mol_type = other DNA
gaccccaagaagaagaggaaggtggaccccaagaagaagaggaaggtggaccccaagaagaagaggaaggtg

SEQ ID NO: 15 - DNA, 30 bp, synthetic construct
  source 1..30: mol_type = other DNA
ctgcctcccctggagcgcctgaccctggac

SEQ ID NO: 16 - DNA, 108 bp, synthetic construct
  source 1..108: mol_type = other DNA
gcggccgccaccatgtacccgtatgatgttccggattacgctggctatccctacgacgtgcccgactatgccgggtacccctatgacgtcccagactacgcagctagc

SEQ ID NO: 17 - DNA, 69 bp, synthetic construct
  source 1..69: mol_type = other DNA
aagcttgcggccgccaccatgggcaagcccatccccaaccccctgctgggcctggacagcaccgctagc

SEQ ID NO: 18 - DNA, 32 bp, synthetic construct
  source 1..32: mol_type = other DNA
ccccccaagcttatggccgtgtccttcatcgg

SEQ ID NO: 19 - DNA, 31 bp, synthetic construct
  source 1..31: mol_type = other DNA
ccccccaagcttcagagctgccattgtaggg

SEQ ID NO: 20 - DNA, 32 bp, synthetic construct
  source 1..32: mol_type = other DNA
ccccccgcggccgcagagctgccattgtaggg

SEQ ID NO: 21 - DNA, 1029 bp, synthetic construct
  source 1..1029: mol_type = other DNA
atgaccaacttctataaagtttgcctggccgtttttattctggtgtgttgtaatattagccatgcagccgttagctttattggtagcaccgaaaatgatgttggtccgagccagggtagctatagcagcacccatgcaatggataatctgccgtttgtgtataacaccggctataacattggttatcagaatgcaaatgtgtggcgtattagcggtggtttttgtgttggtctggatggtaaagttgatctgccggttgttggtagcctggatggtcagagcatttatggtctgaccgaagaagtgggtctgctgatttggatgggtgataccaattatagccgtggcaccgcaatgagcggtaatagctgggaaaatgtttttagcggttggtgcgttggtaattatgttagcacccagggtctgagcgttcatgttcgtccggttattctgaaacgtaatagcagcgcacagtatagcgttcagaaaaccagcattggtagtattcgtatgcgtccgtataatggtagcagtgcaggtagcgtgcagaccaccgtgaattttagcctgaatccgtttacactgaatgataccgttaccagctgtcgtctgctgaccccgagcgcagttaatgttagcctggcagcaattagcgcaggtcagctgccgagcagcggtgatgaagttgttgcaggtacaaccagcctgaaactgcagtgtgatgcgggtgttaccgtttgggcaaccctgaccgatgcaaccacaccgagcaatcgtagcgatattctgaccctgacaggtgcaagcaccgcaacaggtgttggcctgcgtatttacaaaaataccgatagcacaccgctgaaatttggtccggatagtccggttaaaggtaatgaaaatcagtggcagctgagcaccggcaccgaaaccagtccgagcgttcgtctgtatgttaaatatgtgaatacaggcgaaggcattaatccgggtacagttaatggtattagcacctttaccttcagctaccag

SEQ ID NO: 22 - protein, 444 aa, synthetic construct
MAVSFIGSTENDVGPSQGSYSSTHAMDNLPFVYNTGYNIGYQNANVWRISGGFCVGLDGKVDLPVVGSLDGQSIYGLTEEVGLLIWMGDTNYSRGTAMSGNSWENVFSGWCVGNYVSTQGLSVHVRPVILKRNSSAQYSVQKTSIGSIRMRPYNGSSAAATMGKPIPNPLLGLDSTASIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEKLPPLERLTL

SEQ ID NO: 23 - protein, 473 aa, synthetic construct
MAVSFIGSTENDVGPSQGSYSSTHAMDNLPFVYNTGYNIGYQNANVWRISGGFCVGLDGKVDLPVVGSLDGQSIYGLTEEVGLLIWMGDTNYSRGTAMSGNSWENVFSGWCVGNYVSTQGLSVHVRPVILKRNSSAQYSVQKTSIGSIRMRPYNGSSAAATMYPYDVPDYAGYPYDVPDYAGYPYDVPDYAASIPLLNAKQILGQLDGGSVAVLPVVDSTNQYLLDRIGELKSGDACIAEYQQAGRGSRGRKWFSPFGANLYLSMFWRLKRGPAAIGLGPVIGIVMAEALRKLGADKVRVKWPNDLYLQDRKLAGILVELAGITGDAAQIVIGAGINVAMRRVEESVVNQGWITLQEAGINLDRNTLAAMLIRELRAALELFEQEGLAPYLSRWEKLDNFINRPVKLIIGDKEIFGISRGIDKQGALLLEQDGVIKPWMGGEISLRSAEKPKKKRKVDPKKKRKVDPKKKRKV

SEQ ID NO: 24 - DNA, 1440 bp, synthetic construct
  source 1..1440: mol_type = other DNA
atggccgtgtccttcatcggcagcaccgaaaatgatgtgggccctagccagggcagctacagctctacacacgccatggacaacctgcctttcgtgtacaacaccggctacaatatcggctaccagaacgccaacgtgtggcggatctctggcggcttttgtgttggcctggacggcaaagtggatctgcctgttgtgggctctctggacggccagtctatctacggcctgacagaggaagtgggcctgctgatctggatgggcgacaccaattacagcagaggcacagccatgagcggcaacagctgggagaatgtgttcagcggatggtgcgtgggcaactacgtgtccacacagggactgtctgtgcacgtgcggcctgtgatcctgaagagaaatagcagcgcccagtacagcgtgcagaaaaccagcatcggctccatcagaatgcggccctacaatggcagctctgcggccgccaccatgtacccgtatgatgttccggattacgctggctatccctacgacgtgcccgactatgccgggtacccctatgacgtcccagactacgcagctagcatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctatgctgatccgagagctgcgggcagccctggaactgttcgagcaggaaggcctggctccatatctgtcacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaaggaattcagcagggccgaccccaagaagaagaggaaggtggaccccaagaagaagaggaaggtggaccccaagaagaagaggaaggtgtga

SEQ ID NO: 25 - DNA, 1350 bp, synthetic construct
  source 1..1350: mol_type = other DNA
atggccgtgtccttcatcggcagcaccgaaaatgatgtgggccctagccagggcagctacagctctacacacgccatggacaacctgcctttcgtgtacaacaccggctacaatatcggctaccagaacgccaacgtgtggcggatctctggcggcttttgtgttggcctggacggcaaagtggatctgcctgttgtgggctctctggacggccagtctatctacggcctgacagaggaagtgggcctgctgatctggatgggcgacaccaattacagcagaggcacagccatgagcggcaacagctgggagaatgtgttcagcggatggtgcgtgggcaactacgtgtccacacagggactgtctgtgcacgtgcggcctgtgatcctgaagagaaatagcagcgcccagtacagcgtgcagaaaaccagcatcggctccatcagaatgcggccctacaatggcagctctaagcttgcggccgccaccatgggcaagcccatccccaaccccctgctgggcctggacagcaccgctagcatcccgctgctgaacgctaaacagattctgggacagctggacggcgggagcgtggcagtcctgcctgtggtcgactccaccaatcagtacctgctggatcgaatcggcgagctgaagagtggggatgcttgcattgcagaatatcagcaggcagggagaggaagcagagggaggaaatggttctctccttttggagctaacctgtacctgagtatgttttggcgcctgaagcggggaccagcagcaatcggcctgggcccggtcatcggaattgtcatggcagaagcgctgcgaaagctgggagcagacaaggtgcgagtcaaatggcccaatgacctgtatctgcaggatagaaagctggcaggcatcctggtggagctggccggaataacaggcgatgctgcacagatcgtcattggcgccgggattaacgtggctatgaggcgcgtggaggaaagcgtggtcaatcagggctggatcacactgcaggaagcagggattaacctggacaggaatactctggccgctatgctgatccgagagctgcgggcagccctggaactgttcgagcaggaaggcctggctccatatctgtcacggtgggagaagctggataacttcatcaatagacccgtgaagctgatcattggggacaaagagattttcgggattagccgggggattgataaacagggagccctgctgctggaacaggacggagttatcaaaccctggatgggcggagaaatcagtctgcggtctgccgaaaagctgcagctgcctcccctggagcgcctgaccctggactaa

SEQ ID NO: 26 - protein, 5 aa, synthetic construct
GGGGS

SEQ ID NO: 27 - protein, 30 aa, synthetic construct
GGGGSGGGGSGGGGSGGGGSGGGGSGGGGS

SEQ ID NO: 28 - protein, 8 aa, synthetic construct
DYKDDDDK

(19) United States
(12) Patent Application Publication
(10) Pub. No.: US 2025/0003977 A1
(43) Pub. Date: Jan. 2, 2025

(54) INTRACELLULAR GLYCAN PROXIMITY LABELING METHODS AND APPLICATIONS THEREOF

(71) Applicant: Wayne State University, Detroit, MI (US)

(72) Inventors: Charlie Fehl, Detroit, MI (US); Yimin Liu, Detroit, MI (US); Zachary M. Nelson, Detroit, MI (US)

(21) Appl. No.: 18/760,890

(22) Filed: Jul. 1, 2024

Related U.S. Application Data
(60) Provisional application No. 63/524,346, filed on Jun. 30, 2023.

(51) Int. Cl. G01N 33/68; C07K 14/47; C07K 14/705; C07K 16/44; C12N 9/00

(52) U.S. Cl. CPC G01N 33/6842; C07K 14/47; C07K 14/705; C07K 14/7056; C07K 16/44; C12N 9/93; C12Y 603/04015; G01N 33/6848; G01N 2333/9015; G01N 2440/32

(57) ABSTRACT

The present disclosure relates to method of detecting proteins proximal to a target protein using fusion proteins which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein. Such methods include contacting a living cell with the fusion protein under compatible biological conditions, whereby the fusion protein specifically binds to a glycosylation post-translational modification of a target protein of the cell; providing biotin to the living cell, whereby the mutant E. coli biotin ligase BirA ligates biotin to proteins proximal to the target protein; and detecting the biotinylated proteins, thereby detecting proteins proximal to the target protein.

### REFERENCE TO RELATED APPLICATION

This application claims priority from U.S. Provisional Patent Application Ser. No. 63/524,346, filed Jun. 30, 2023, the entire content of which is incorporated herein by reference.

### GOVERNMENT SUPPORT

This invention was made with government support under R35GM142637 awarded by the National Institute of General Medicine Sciences. The government has certain rights in the invention.

### SEQUENCE LISTING

The instant application contains a Sequence Listing which has been submitted electronically as a file in XML format and is hereby incorporated by reference in its entirety. Said XML format file, created on Jul. 1, 2024, is named 47WAY16102 Sequence listing.xml and is 32,699 bytes in size.

### FIELD OF THE INVENTION

According to general aspects, the present disclosure relates to intracellular glycan proximity labeling methods and applications thereof. According to specific aspects, the present disclosure relates to fusion proteins which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein.

### BACKGROUND OF THE INVENTION

A fundamental mechanism that all eukaryotic cells use to adapt to their environment is dynamic protein modification with monosaccharide sugars. In humans, O-linked N-acetylglucosamine (O-GlcNAc) is rapidly added to and removed from diverse protein sites as a response to fluctuating nutrient levels, stressors, and signaling cues.

The O-GlcNAc (O-linked N-acetylglucosamine) modification on proteins is a nutrient- and condition-sensing post-translational modification essential for all mammalian cells to adapt to their microenvironment. Thousands of O-GlcNAc sites regulate cell biology, including signaling and transcription, in both nutrient-driven and nutrient-independent roles. Protein O-GlcNAcylation is cycled by two proteins, O-GlcNAc transferase (OGT) and O-GlcNAcase (OGA) (FIG. 2A). The OGT gene can produce three isoforms, each of which is most active in a distinct cellular location: nucleocytoplasmic ncOGT is primarily found in the nucleus; mitochondrial mOGT is found in mitochondria; and short sOGT, which lacks a nuclear localization signal and is therefore mainly cytosolic. During insulin signaling, OGT is known to move to the plasma membrane, where it is then active on membrane proteins. Therefore, a crucial facet of O-GlcNAc regulation depends on the spatial location of target proteins in the cell and which isoform(s) of OGT is produced at a given time (FIG. 2B).

A second mechanism for O-GlcNAc regulation is time-based because O-GlcNAc modifications can be dynamically removed by OGA. In this vein, mammalian cells regulate the balance of OGT/OGA concerning overall O-GlcNAc levels, employing a variety of mechanisms including regulatory modifications, expression, as well as levels of OGT and OGA pre-mRNA transcripts. In particular, this mRNA regulation via alternative splicing enables cells to respond to O-GlcNAc perturbations within 30 min During OGT/OGA rebalancing, O-GlcNAc events in this 30 min phase are increasingly recognized as critical for a wide range of cellular functions.

There is a continuing need for a compositions and methods specific for O-GlcNAc sugar modifications which allow detection of changes in space and time.

### SUMMARY OF THE INVENTION

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the glycan binding component is selected from the group consisting of: a lectin, a collectin, a ficolin, a C-reactive protein, and a carbohydrate-binding domain of any thereof.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the glycan binding component is selected from the group consisting of: an aptamer, an antibody, and an antigen-binding fragment of an antibody.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the glycan binding component is GafD lectin.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the mutant E. coli biotin ligase BirA comprises SEQ ID NO:2, or a variant thereof having enzymatic activity to ligate biotin to proteins proximal to the target protein.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the mutant E. coli biotin ligase BirA comprises SEQ ID NO:2, or a variant thereof having enzymatic activity to ligate biotin to proteins proximal to the target protein, and wherein the glycan binding component is selected from the group consisting of: a lectin, a collectin, a ficolin, a C-reactive protein, and a carbohydrate-binding domain of any thereof.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the mutant E. coli biotin ligase BirA comprises SEQ ID NO:2, or a variant thereof having enzymatic activity to ligate biotin to proteins proximal to the target protein, and wherein the glycan binding component is selected from the group consisting of: an aptamer, an antibody, and an antigen-binding fragment of an antibody.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the mutant E. coli biotin ligase BirA comprises SEQ ID NO:2, or a variant thereof having enzymatic activity to ligate biotin to proteins proximal to the target protein, and wherein the glycan binding component is GafD lectin.

According to aspects of the present disclosure, the glycan binding component has a C-terminus and an N-terminus, the mutant E. coli biotin ligase BirA has a C-terminus and an N-terminus, and the C-terminus of the glycan binding component is linked to the N-terminus of the mutant E. coli biotin ligase BirA.

According to aspects of the present disclosure, the glycan binding component is linked to the mutant E. coli biotin ligase BirA by a linker disposed between the glycan binding component and the mutant E. coli biotin ligase BirA.

A localization signal peptide is included in a fusion protein according to aspects of the present disclosure. According to aspects of the present disclosure, the localization signal peptide is capable of promoting localization of the fusion protein to a subcellular compartment selected from the group consisting of: nucleus, cytosol, mitochondria, endoplasmic reticulum, and plasma membrane.

An exogenous detectable tag is included in a fusion protein according to aspects of the present disclosure.

Methods of detecting proteins proximal to a target protein are provided according to aspects of the present disclosure which include: contacting a living cell with a fusion protein of the present disclosure under compatible biological conditions, whereby the fusion protein specifically binds to a glycosylation post-translational modification of a target protein of the cell; providing biotin to the living cell, whereby the mutant E. coli biotin ligase BirA ligates biotin to proteins proximal to the target protein; and detecting the biotinylated proteins, thereby detecting proteins proximal to the target protein.

Methods of detecting proteins proximal to a target protein are provided according to aspects of the present disclosure wherein detecting the biotinylated proteins comprises purifying the biotinylated proteins and detecting the purified biotinylated proteins.

Methods of detecting proteins proximal to a target protein are provided according to aspects of the present disclosure wherein detecting the purified biotinylated proteins comprises mass spectrometry.

Methods of detecting proteins proximal to a target protein are provided according to aspects of the present disclosure wherein detecting the purified biotinylated proteins comprises chromatography. According to aspects of the present disclosure, the chromatography comprises gel electrophoresis. According to aspects of the present disclosure, the chromatography comprises gel electrophoresis and transfer of the electrophoresed purified biotinylated proteins to a membrane.

According to aspects of the present disclosure, contacting the living cell with the fusion protein comprises introducing an expression construct encoding the fusion protein into the cell.

Expression constructs are provided according to aspects of the present disclosure which include a nucleic acid encoding a fusion protein wherein the fusion protein includes: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein.

Cells are provided according to aspects of the present disclosure which include an expression construct which includes a nucleic acid encoding a fusion protein wherein the fusion protein includes: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein.

### BRIEF DESCRIPTION OF THE DRAWINGS

FIG. 1 is a schematic diagram of aspects of O-GlcNAc post-translational modification.

FIGS. 2A, 2B, 2C, and 2D: Protein O-GlcNAc post-translational modifications allow cells to rapidly adapt to changes in their environments, requiring intracellular tools for functional labeling. FIG. 2A is an illustration showing that O-GlcNAc modifications are cycled by two enzymes on 1000s of known substrates, occurring at varying timescales and driven by fluctuations in nutrients, cell stressors, or signaling cues. FIG. 2B is an illustration showing that OGT isoforms localize to and move between subcellular compartments, enabling spatiotemporal control of protein functions in cells. FIG. 2C is a diagramatic illustration of a GlycoID system according to aspects of the present disclosure for live-cell O-GlcNAc labeling. The GlcNAc-binding lectin GafD localized a proximity labeling enzyme TurboID to O-GlcNAcylated proteins. Treatment with the TurboID substrate (biotin)-labeled proximal proteins in ca. 10 nm radius. Subcellular targeting enabled spatial labeling. Changes in cellular O-GlcNAc proteins and proximal interactomes were obtained in live cells under serum or insulin stimulation. FIG. 2D shows comparison of two reported O-GlcNAc-active proximity labeling systems, OGA-BioID (ref 20), and the substrate-recognition tetratricopeptide repeat region (TPR) domain of OGT, OGT-TRP-BioID (ref 21). The complete lists of proteins share modest overlap (11-16%), but each label has distinct sets of O-GlcNAc-related protein interactomes. It is noted that GlycoID and OGT-TPR-BioID were performed in HeLa cells and OGA-BioID is reported in HepG2 cells so this comparison could be affected by cell type-specific proteome differences.

FIGS. 3A, 3B, and 3C: Confirmation of GlycoID labeling. FIG. 3A diagrammatically shows construct design for the GlycoID O-GlcNAc proximity labeling systems according to aspects of the present disclosure. cyt-mTurbo and nuc-mTurbo are controls without a glycan binding component (undirected control). By contrast, cyt-GlycoID and nucGlycoID include a glycan binding component (GafD lectin) and a mutant E. coli biotin ligase BirA having enzymatic activity to ligate biotin to proteins proximal to the target protein (mTurbo, also called miniTurbo), i.e. GlcNAc-directed systems. cyt=cytosol targeting and nuc=nucleus targeting. Immunofluorescence detection confirmed correct cellular location for each construct. FIG. 3B shows immunoblots demonstrating expression of the indicated constructs in Hela cells with V5 and HA used as epitope tags for immunoblotting. FIG. 3C shows results in a global O-GlcNAc blot following expression of GlycoID constructs for 48 hours in HeLa cells.

FIGS. 4A and 4B: Comparative proteomics with GlycoID constructs. FIG. 4A diagrammatically shows exclusive hits between GlcNAc-binding nuc-GlycoID and control nuc-mTurbo, 6 hours at 100 μM biotin labeling. FIG. 4B shows analysis for cyt-GlycoID versus cyt-mTurbo.

FIGS. 5A and 5B: Functional O-GlcNAc proteomics with GlycoID during insulin signaling. FIG. 5A diagrammatically shows exclusive hits between serum-starved nuc-GlycoID and insulin-stimulated nuc-GlycoID, 30 min at 500 μM biotin labeling. FIG. 5B shows results of a similar analysis for cyt-GlycoID-starved versus cyt-GlycoID+insulin.

FIG. 6: is an image of a Western blot for the expression of cyt-mTurboID (1, 2), cyt-GlycoID (3, 4), nuc-mTurboID (5, 6), nuc-GlycoID (7, 8) in HEK293T cells. All experiments used 100 μM biotin and were allowed to label for 6 hours. Expected sizes: cyt-mTurbo, 31.2 kDa; cyt-GlycoID, 48.6 kDa; nuc-mTurbo, 35.3 kDa; nuc-GlycoID, 52.5 kDa.

FIGS. 7A, 7B, 7C, and 7D: show results of extended immunoblot studies for siRNA studies and GlycoID labeling. Western blot analyses for the activity of nuc-mTurbo, and nuc-GlycoID blotted against O-GlcNAc MultiMab (1:1000)/Anti-Rabbit-AlexaFluor-488 (1:1000), and Streptavidin-Cy5 (1:1000) were performed under knockdown conditions specified herein. Western blot analyze for the activity of cyt-mTurbo, and cyt-GlycoID blotted against O-GlcNAc MultiMab (1:1000)/Anti-Rabbit-AlexaFluor-488 (Green, 1:1000) and Streptavidin-Cy5 (Red, 1:1000) were performed under knockdown conditions specified herein. Experiments for these blots used 100 μM biotin with 6 hours allowed for labeling. Blots were imaged using the iBright™ FL1500 instrument. FIG. 7A shows results of densitometry analysis of OGT bands vs. GAPDH loading control. FIG. 7B shows results of densitometry analysis of OGA bands vs. GAPDH loading control. FIG. 7C shows results of densitometry analysis of construct expression bands vs. GAPDH loading control. FIG. 7D shows results of densitometry analysis of biotin bands (Cy5 signal) vs. normalized expression patterns for each construct.

FIGS. 8A and 8B: Analysis of nuc-mTurbo and cyt-mTurbo (non-sugar targeted) quantitative proteomics with GlycoID constructs. FIG. 8A diagrammatically shows exclusive hits between non-targeted nuc-mTurbo vs. nuc-GlycoID and lists the protein groups labeled by nuc-GlycoID. FIG. 8B) diagrammatically shows results of analysis for cyt-GlycoID vs. cyt-mTurbo. Enrichment analysis was performed between nuc-mTurbo and nuc-GlycoID and showed statistically significant hits above the volcano plot cutoffs. Physical interactions between nuc-GlycoID hits revealed functional clusters with key O-GlcNAc linkages.

FIGS. 9A and 9B: Analysis of GlycoID constructs between starved and serum fed conditions. FIG. 9A diagrammatically shows exclusive hits between starved nuc-GlycoID vs. 10% fetal bovine serum (FBS)-supplemented nuc-GlycoID and lists the protein groups labeled by nuc-GlycoID serum-fed hits. FIG. 9B diagrammatically shows results of analysis for starved cyt-GlycoID vs. 10% FBS-supplemented cyt-GlycoID. Enrichment analysis between starved and fed conditions showed statistically significant hits above the volcano plot cutoffs. Physical interactions between serum-fed hits revealed functional clusters with key O-GlcNAc linkages.

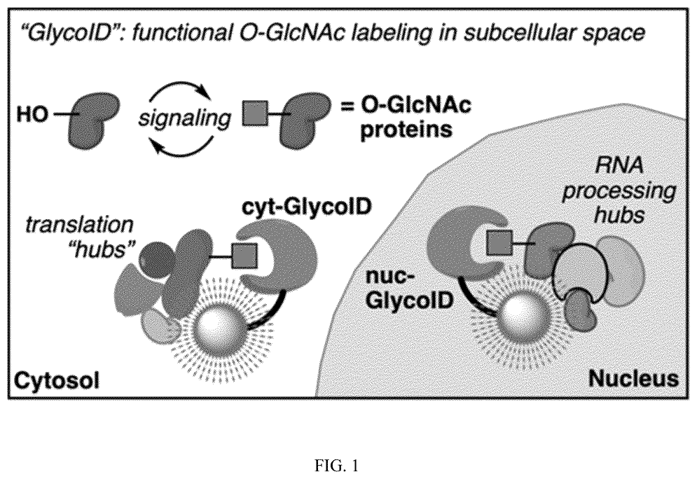

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; CRISPR/Cas: A Laboratory Manual, Doudna and Mali (eds), Cold Spring Harbor Laboratory Press, Cold Spring Harbor, NY, USA, 2016; D. L. Nelson and M. M. Cox, Lehninger Principles of Biochemistry, 4th Ed., W.H. Freeman & Company, 2004; J.-H. Fuhrhop et al. (Eds.), Organic Synthesis, Concepts and Methods, 3rd Ed., Wiley-VCH Cerlag GmbH & Co. KGaA, 2003; Herdewijn, P. (Ed.), Oligonucleotide Synthesis: Methods and Applications, Methods in Molecular Biology, Humana Press, 2004; D. J. Taxman (ed.), siRNA Design, Methods and Protocols, Humana Press, 2012; Harlow, E. and Lane, D., Antibodies: A Laboratory Manual, Cold Spring Harbor Laboratory Press, 1988; J. D. Pound (Ed.) Immunochemical Protocols, Methods in Molecular Biology, Humana Press, 2nd ed., 1998; Chu, E. and Devita, V. T., Eds., Physicians' Cancer Chemotherapy Drug Manual, Jones & Bartlett Publishers, 2021; J. M. Kirkwood et al., Eds., Current Cancer Therapeutics, 4th Ed., Current Medicine Group, 2001; A Adejare (Ed.), Remington: The Science and Practice of Pharmacy, Elsevier, 23rd Ed., 2021; L. V. Allen, Jr. et al., Ansel's Pharmaceutical Dosage Forms and Drug Delivery Systems, 11th Ed., Wolters Kluwer, 2016; and L. Brunton et al., Goodman & Gilman's The Pharmacological Basis of Therapeutics, McGraw-Hill Education, 13th Ed., 2018.

The singular terms “a,” “an,” and “the” are not intended to be limiting and include plural referents unless explicitly stated otherwise or the context clearly indicates otherwise.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

The term “about” as used herein in reference to a number is used herein to include numbers which are greater, or less than, a stated or implied value by 1%, 5%, 10%, or 20%.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

The terms “first,” “second,” and the like are used herein to describe various features or elements, but these features or elements are not intended to be limited by these terms, but are only used to distinguish one feature or element from another feature or element. Thus, a first feature or element could be termed a second feature or element, and vice versa, without departing from the teachings of the present disclosure.

Fusion proteins are provided according to aspects of the present disclosure which include: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein.

The term “glycan binding component” as used herein refers to a binding agent characterized by specific binding to a specified glycan target. The phrase “specific binding” and grammatical equivalents as used herein in reference to binding of a binding agent to a specified glycan target refers to binding of the binding agent to the specified glycan target without substantial binding to other substances present in a cell which include a fusion protein according to aspects of the present disclosure. The term “binding” refers to a physical or chemical interaction between a binding agent and its target. Binding includes, but is not limited to, ionic bonding, non-ionic bonding, covalent bonding, hydrogen bonding, hydrophobic interaction, hydrophilic interaction, and Van der Waals interaction.

Specific binding refers to a binding agent that binds to a specified glycan target with greater affinity, greater avidity, and/or greater duration, than to other substances. According to aspects of the present disclosure, a binding agent specifically binds to its glycan target when it has an equilibrium dissociation constant, K_{D}, for its target in the range of about 10-4 to about 10-12, i.e. a K_{D} of about 10-4, about 10-5, about 10-6, about 10-7, about 10-8, about 10-9, about 10-10, about 10-11, or about 10-12. Binding affinity of a binding agent can be determined by Scatchard analysis such as described in P. J. Munson and D. Rodbard, Anal. Biochem., 107:220-239, 1980 or by other methods such as Biomolecular Interaction Analysis using plasmon resonance.

Binding agents specific for a specified glycan target may be obtained from commercial sources or generated for use in methods of the present disclosure according to well-known methodologies.

According to aspects of the present disclosure, the glycan binding component is a binding agent which is, or includes, a lectin, a collectin, a ficolin, a C-reactive protein, or a carbohydrate-binding domain of any thereof. According to aspects of the present disclosure, the glycan binding component is a binding agent which is or includes, an aptamer, antibody, or an antigen-binding fragment of an antibody.

The term “antibody”′ is used herein in its broadest sense and includes antibodies, and antigen-binding fragments, characterized by specific binding to an antigen. An antibody included in methods according to aspects of the present disclosure may be a polyclonal antibody, a monoclonal antibody, a chimeric antibody, a humanized antibody, and/or an antigen-binding antibody fragment of any thereof. An antibody included in methods in particular aspects of the present disclosure includes a standard intact immunoglobulin having four polypeptide chains including two heavy chains (H) and two light chains (L) linked by disulfide bonds. An antibody included in methods in particular aspects of the present disclosure includes an antigen-binding antibody fragments illustratively include an Fab fragment, an Fab′ fragment, an F (ab′) 2 fragment, an Fd fragment, an Fv fragment, an scFv fragment and a domain antibody (dAb), for example. In addition, the term antibody refers to antibodies of various classes including IgG, IgM, IgA, IgD and IgE, as well as subclasses, illustratively including for example human subclasses IgG1, IgG2, IgG3 and IgG4 and marine subclasses IgG1, IgG2, IgG2a. IgG2b, IgG3 and IgGM, for example.

In particular embodiments, an antibody which is characterized by specific binding to its target has a dissociation constant in the range of about 10-4 to about 10-12, i.e. a K_{D} of about 10-4, about 10-5, about 10-6, about 10-7, about 10-8, about 10-9, about 10-10, about 10-11, or about 10-12. Binding affinity of an antibody can be determined by Scatchard analysis such as described in P. J. Munson and D. Rodbard, Anal. Biochem., 107:220-239, 1980 or by other methods such as Biomolecular Interaction Analysis using plasmon resonance. Antibodies may be tested for specific binding to the target by methods illustratively including ELISA, Western blot, and immunocytochemistry.

Antibodies, antigen-binding fragments, and methods for their generation are known in the art, for instance, as described in Antibody Engineering, Kontemann, R. and Dubel, S. (Eds.), Springer, 2001; Harlow, E. and Lane, D., Antibodies: A Laboratory Manual, Cold Spring Harbor Laboratory Press, 1988; Ausubel. F. et al., (Eds.), Short Protocols in Molecular Biology, Wiley, 2002; J. D. Pound (Ed.) Immunochemical Protocols, Methods in Molecular Biology, Humana Press, 2nd ed., 1998; B. K. C. Lo (Ed.), Antibody Engineering: Methods and Protocols, Methods in Molecular Biology, Humana Press, 2003; and Kohler, G. and Milstein, C., Nature, 256:495-497 (1975).

A binding agent according to aspects of the present disclosure may be an aptamer. The term “aptamer” refers to a nucleic acid or peptide that substantially specifically binds to a specified substance. In the case of a nucleic acid aptamer, the aptamer is characterized by binding interaction with a target other than Watson/Crick base pairing or triple helix binding with a second and/or third nucleic acid. Such binding interaction may include Van der Waals interaction, hydrophobic interaction, hydrogen bonding and/or electrostatic interactions, for example. Techniques for identification and generation of aptamers is known in the art as described, for example, in F. M, Ausubel et al., Eds., Short Protocols in Molecular Biology, Current Protocols, Wiley, 2002; S. Klussman, Ed., The Aptamer Handbook: Functional Oligonucleotides and Their Applications, Wiley, 2006; and J. Sambrook and D. W. Russell, Molecular Cloning: A Laboratory Manual, Cold Spring Harbor Laboratory Press, 3rd Ed., 2001.

According to aspects of the present disclosure, the glycan binding component capable of specific binding to a glycosylation post-translational modification of a target protein is a lectin.

According to aspects of the present disclosure, the glycan binding component capable of specific binding to a glycosylation post-translational modification of a target protein is a GafD lectin, or a glycan specific binding fragment thereof. GafD is E. coli N-acetyl-D-glucosamine-specific fimbrial lectin (adhesin) protein GafD is a protein which specifically binds to N-acetyl-D-glucosamine.

According to aspects of the present disclosure, an included GafD lectin includes the following sequence:

(SEQ ID NO: 3)
MTNFYKVCLAVFILVCCNISHAAVSFIGSTENDVGPSQGSYSSTH
AMDNLPFVYNTGYNIGYQNANVWRISGGFCVGLDGKVDLPVVGSL
DGQSIYGLTEEVGLLIWMGDTNYSRGTAMSGNSWENVFSGWCVGN
YVSTQGLSVHVRPVILKRNSSAQYSVQKTSIGSIRMRPYNGSSAG
SVQTTVNFSLNPFTLNDTVTSCRLLTPSAVNVSLAAISAGQLPSS
GDEVVAGTTSLKLQCDAGVTVWATLTDATTPSNRSDILTLTGAST
ATGVGLRIYKNTDSTPLKFGPDSPVKGNENQWQLSTGTETSPSVR
LYVKYVNTGEGINPGTVNGISTFTFSYQ

or a variant thereof.

GlcNAc-binding lectin GafD is described in detail in Saarela S et al., Infect. Immun 1996, 64, 2857-2860, PubMed: 8698525

GafD is selective for GlcNAc-linked molecules over other sugars, including >10-fold binding selectivity over glucose-linked molecules, >100-fold selectivity over GalNAc, and no detectable binding against mannose, fucose, galactose, or sialic acid sugars as detailed in Hsu K-L et al., Mol. BioSyst 2008, 4, 654-662, PubMed: 18493664

A glycan specific binding fragment of GafD lectin is included according to aspects of the present disclosure.

According to aspects of the present disclosure, an included glycan specific binding fragment of GafD lectin includes the following sequence:

(SEQ ID NO: 4)
MAVSFIGSTENDVGPSQGSYSSTHAMDNLPFVYNTGYNIGYQNAN
VWRISGGFCVGLDGKVDLPVVGSLDGQSIYGLTEEVGLLIWMGDT
NYSRGTAMSGNSWENVFSGWCVGNYVSTQGLSVHVRPVILKRNSS
AQYSVQKTSIGSIRMRPYNGSS

or a variant thereof.

Amino acid sequences and nucleic acid sequences are shown or described herein. Methods and compositions of the present invention are not limited to particular amino acid sequences and nucleic acid sequences identified herein and variants of a reference amino acid or nucleic acid sequence are encompassed.

As used herein, the term “variant” defines either an isolated naturally occurring mutant of a protein or nucleic acid, or a recombinantly prepared mutant of a protein or nucleic acid, each of which contain one or more mutations compared to a corresponding reference sequence, such as a wild-type sequence. For example, such mutations in a protein sequence can be one or more amino acid substitutions, additions, and/or deletions. In a further example, For example, such mutations in a nucleic acid sequence can be one or more nucleotide substitutions, additions, and/or deletions. The term “variant” further refers to orthologues.

The term “wild-type” refers to a naturally occurring, or unmutated, protein or nucleic acid.

According to aspects of the present disclosure, a variant protein includes an amino acid sequence having at least 70%, at least 75%, at least 80%, at least 85%, at least 90%, at least 95%, at least 96%, at least 97%, at least 98%, at least 99%, or greater than 99%, identity with the reference amino acid sequence, and retains at least a substantial proportion (at least about 50%, 60%, 70%, 80%, 90%, 95%, 98%, 99% or more) of the functional characteristics of the reference protein.

According to aspects of the present disclosure, a variant protein includes an amino acid sequence having at least 90%, 91%, 92%, 93%, 94%, 95%, 96%, 97%, 98%, 99% identity, or greater than 99%, identity with the reference amino acid sequence, and retains at least a substantial proportion (at least about 50%, 60%, 70%, 80%, 90%, 95%, 98%, 99% or more) of the functional characteristics of the reference protein.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X100%). The two sequences compared are generally the same length or nearly the same length. Optionally, the two sequences are natural variants of a structural domain of a protein or two related proteins.

The determination of percent identity between two sequences can also be accomplished using a mathematical algorithm. A preferred, non-limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present invention. BLAST protein searches are performed with the XBLAST program parameters set, e.g., to score 50, wordlength=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules (Id.). When utilizing BLAST, Gapped BLAST, and PSI Blast programs, the default parameters of the respective programs (e.g., of XBLAST and NBLAST) are used (see, e.g., the NCBI website). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

One of skill in the art will recognize that one or more nucleotide or amino acid mutations can be introduced without altering the functional properties of a given nucleic acid or protein, respectively.

Mutations can be introduced using standard molecular biology techniques, such as site-directed mutagenesis and PCR-mediated mutagenesis, to produce variants. For example, one or more amino acid substitutions, additions, or deletions can be made without altering the functional properties of a reference protein. When comparing a reference protein to a putative variant, amino acid similarity may be considered in addition to identity of amino acids at corresponding positions in an amino acid sequence. “Amino acid similarity” refers to amino acid identity and conservative amino acid substitutions in a putative variant compared to the corresponding amino acid positions in a reference protein.

Conservative amino acid substitutions can be made or may be present in reference proteins to produce or identify variants.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar/nonpolar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size; alanine, cysteine, aspartate, glycine, asparagine, proline, threonine, serine, valine are all typically considered to be small.

A variant can include synthetic amino acid analogs, amino acid derivatives and/or non-standard amino acids, illustratively including, without limitation, alphaaminobutyric acid, citrulline, canavanine, cyanoalanine, diaminobutyric acid, diaminopimelic acid, dihydroxy-phenylalanine, djenkolic acid, homoarginine,-18-18 hydroxyproline, norleucine, norvaline, 3-phosphoserine, homoserine, 5-hydroxytryptophan, 1-methylhistidine, 3-methylhistidine, and ornithine.

It will be appreciated by those of ordinary skill in the art that, due to the degenerate nature of the genetic code, alternate nucleic acid sequences encode a specified protein such variant nucleic acid sequences may be used in compositions and methods described herein.

Protein variants are encoded by nucleic acids having a high degree of identity with a nucleic acid encoding a corresponding reference protein, such as a wild-type protein, or a corresponding portion thereof. The complement of a nucleic acid encoding a variant specifically hybridizes with a nucleic acid encoding a corresponding reference protein, such as a wild-type protein, under high stringency conditions.

The term “nucleic acid” refers to RNA or DNA molecules having more than one nucleotide in any form including single-stranded, double-stranded, oligonucleotide or polynucleotide. The term “nucleotide sequence” refers to the ordering of nucleotides in an oligonucleotide or polynucleotide in a single-stranded form of nucleic acid.

The term “complementary” refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6×SSC, 5×Denhardt's solution, 30% formamide, and 100 micrograms/ml denatured salmon sperm at 37° C. overnight followed by washing in a solution of 0.1×SSC and 0.1% SDS at 60° C. for 15 minutes. SSC is 0.15M NaCl/0.015M Na citrate. Denhardt's solution is 0.02% bovine serum albumin/0.02% FICOLL/0.02% polyvinylpyrrolidone.

Nucleic acids encoding a protein, or a variant thereof, can be isolated or generated recombinantly or synthetically using well-known methodology.

The term “wild-type E. coli biotin ligase BirA” refers to the protein of SEQ ID NO: 1.

(SEQ ID NO: 1)
MKDNTVPLKLIALLANGEFHSGEQLGETLGMSRAAINKHIQTLRD
WGVDVFTVPGKGYSLPEPIQLLNAKQILGQLDGGSVAVLPVIDST
NQYLLDRIGELKSGDACIAEYQQAGRGRRGRKWFSPFGANLYLSM
FWRLEQGPAAAIGLSLVIGIVMAEVLRKLGADKVRVKWPNDLYLQ
DRKLAGILVELTGKTGDAAQIVIGAGINMAMRRVEESVVNQGWIT
LQEAGINLDRNTLAAMLIRELRAALELFEQEGLAPYLSRWEKLDN
FINRPVKLIIGDKEIFGISRGIDKQGALLLEQDGIIKPWMGGEIS
LRSAEK

The term “mutant E. coli biotin ligase BirA” refers to a mutant version of the protein of SEQ ID NO:1, the mutant E. coli biotin ligase BirA having enzymatic activity to ligate biotin to proteins proximal to the target protein.

The protein of SEQ ID NO:2, or a variant thereof, is a “mutant E. coli biotin ligase BirA” having enzymatic activity to ligate biotin to proteins proximal to the target protein.

SEQ ID NO: 2
MIPLLNAKQILGQLDGGSVAVLPVVDSTNQYLLDRIGELKSGDAC
IAEYQQAGRGSRGRKWFSPFGANLYLSMFWRLKRGPAAIGLGPVI
GIVMAEALRKLGADKVRVKWPNDLYLQDRKLAGILVELAGITGDA
AQIVIGAGINVAMRRVEESVVNQGWITLQEAGINLDRNTLAAMLI
RELRAALELFEQEGLAPYLSRWEKLDNFINRPVKLIIGDKEIFGI
SRGIDKQGALLLEQDGVIKPWMGGEISLRSAEK

The protein of SEQ ID NO:2, or a variant thereof, is a “mutant E. coli biotin ligase BirA” having enzymatic activity to ligate biotin to proteins proximal to the target protein.

SEQ ID NO: 11
IPLLNAKQILGQLDGGSVAVLPVVDSTNQYLLDRIGELKSGDACI
AEYQQAGRGSRGRKWFSPFGANLYLSMFWRLKRGPAAIGLGPVIG
IVMAEALRKLGADKVRVKWPNDLYLQDRKLAGILVELAGITGDAA
QIVIGAGINVAMRRVEESVVNQGWITLQEAGINLDRNTLAAMLIR
ELRAALELFEQEGLAPYLSRWEKLDNFINRPVKLIIGDKEIFGIS
RGIDKQGALLLEQDGVIKPWMGGEISLRSAEK

According to aspects of the present disclosure, the mutant E. coli biotin ligase BirA comprises a variant of SEQ ID NO:1 having enzymatic activity to ligate biotin to proteins proximal to the target protein.

According to aspects of the present disclosure, the mutant E. coli biotin ligase BirA comprises SEQ ID NO:2, or a variant thereof having enzymatic activity to ligate biotin to proteins proximal to the target protein.

According to aspects of the present disclosure, the mutant E. coli biotin ligase BirA comprises SEQ ID NO:11, or a variant thereof having enzymatic activity to ligate biotin to proteins proximal to the target protein.

According to aspects of the present disclosure, the mutant E. coli biotin ligase BirA comprises at least a R118S mutation compared to wild-type E. coli biotin ligase BirA.

According to aspects of the present disclosure, the mutant E. coli biotin ligase BirA comprises at least a R118S mutation and deletion of 62 amino acids from the N-terminus compared to wild-type E. coli biotin ligase BirA.

According to aspects of the present disclosure, the mutant E. coli biotin ligase BirA comprises at least Q65P, 187V, R118S, Q141R, E149K, S150G, L151P, V160A, T192A, K194I, M209V, S236P, M241T, and I305V mutations compared to wild-type E. coli biotin ligase BirA.

According to aspects of the present disclosure, the mutant E. coli biotin ligase BirA comprises at least Q65P, 187V, R118S, Q141R, E149K, S150G, L151P, V160A, T192A, K194I, M209V, S236P, M241T, and 1305V mutations and deletion of 62 amino acids from the N-terminus compared to wild-type E. coli biotin ligase BirA.

According to aspects of the present disclosure, the glycan binding component has a C-terminus and an N-terminus, the mutant E. coli biotin ligase BirA has a C-terminus and an N-terminus, and the C-terminus of the glycan binding component is linked to the N-terminus of the mutant E. coli biotin ligase BirA.

A linker is disposed between, and linked to each of, two components of a fusion protein according to aspects of the present disclosure, thereby linking the two components through the linker.

According to aspects of the present disclosure, an included linker is, or includes about 1 to 100 amino acids, such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids. According to aspects of the present disclosure, is a peptide including about 1 to 100 amino acids, such as about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids.

According to aspects of the present disclosure, the linker can be, or include, a bond, an atom, a multi-atom group, or a chain of atoms. Non-limiting examples of a linker which is an atom are oxygen, and sulfur. A non-limiting example of a linker which is a multi-atom group is C (O).

According to aspects of the present disclosure, an included linker is, or includes, a chain of atoms such as, branched or linear chain of 2-20, or more, atoms. According to aspects of the present disclosure, a linker is, or includes, a linear chain of 3-20, or more, atoms. According to aspects of the present disclosure, a linker is, or includes, a linear chain of 3, 4, 5, 6, 7, 8, 9, 10, 11, 12, 13, 14, 15, 16, 17, 18, 19, or 20 atoms. According to aspects of the present disclosure, a linker is, or includes, a linear chain of 6, 7, 8, 9, 10, 11, or 12 atoms. According to aspects of the present disclosure, a linker is, or includes, a linear chain of 8, 9, 10, 11, 12, 13, 14, 15, 16, 17, 18, 19, or 20 atoms. According to aspects of the present disclosure, a linker is, or includes, a linear chain of 10, 11, 12, 13, 14, 15, 16, 17, 18, 19, or 20 atoms. According to aspects of the present disclosure, a linker is, or includes, a linear chain of 10, 11, 12, 13, 14, or 15 atoms.

According to aspects of the present disclosure, an included linker is, or includes, a chain of atoms such as, but not limited to, substituted or unsubstituted C₁-C₂₀ alkyl, substituted or unsubstituted C₂-C₂₀ alkenyl, substituted or unsubstituted C₂-C₂₀ alkynyl, substituted or unsubstituted C₆-C₁₂ aryl, substituted or unsubstituted C₃-C₁₂ cycloalkyl, substituted or unsubstituted C₅-C₁₂ heteroaryl, or substituted or unsubstituted C₅-C₁₂ heterocyclyl. According to aspects of the present disclosure, the chain includes at least one hydroxyl functional group, and preferably at least one terminal hydroxyl functional group. The term “terminal hydroxyl functional group” as used herein refers to a hydroxyl group on the final atom of a chain of atoms in a linker, i.e. the atom of the chain of atoms which is furthest from the magnetic particle, or within no more than 2, 3, or 4 atoms from the final atom of the chain of atoms in a linker.

According to aspects of the present disclosure, a linker is disposed between, and linked to each of, a glycan binding component and a mutant E. coli biotin ligase BirA, thereby linking the glycan binding component and the mutant E. coli biotin ligase BirA through the linker.

According to aspects of the present disclosure, the glycan binding component is linked to the mutant E. coli biotin ligase BirA by a linker disposed between the glycan binding component and the mutant E. coli biotin ligase BirA.

According to aspects of the present disclosure, the fusion protein includes a localization signal peptide. According to aspects of the present disclosure, the localization signal peptide is capable of promoting localization of the fusion protein to a subcellular compartment selected from the group consisting of: nucleus, cytosol, mitochondria, endoplasmic reticulum, and plasma membrane. One or more localization signal peptides are included in a fusion protein according to aspects of the present disclosure. When two or more localization signal peptides are included in a fusion protein according to aspects of the present disclosure, a linker is optionally disposed between the two or more localization signal peptides.

According to aspects of the present disclosure, the localization signal peptide is a mitochondrial localization signal. An exemplary mitochondrial localization signal is

(SEQ ID NO: 5)
MLGFVGRVAAAPASGALRRLTPSASLPPAQLLLRAAPTAVHPVRDYA,

or a variant thereof.

According to aspects of the present disclosure, the localization signal peptide is a plasma membrane localization signal. An exemplary plasma membrane localization signal is MGCINSKRK (SEQ ID NO:6), or a variant thereof.

According to aspects of the present disclosure, the localization signal peptide is a nuclear localization signal. An exemplary nuclear localization signal (NLS) is PKKKRKV (SEQ ID NO:7), or a variant thereof.

According to aspects of the present disclosure, the localization signal peptide is a cytoplasm localization signal. An exemplary cytoplasm localization signal (NES) is LPPLERLTL (SEQ ID NO:8), or a variant thereof.

According to aspects of the present disclosure, the fusion protein includes an exogenous detectable tag, such as a V5 tag or HA tag, or a variant of either thereof. An exemplary V5 tag is GKPIPNPLLGLDST (SEQ ID NO:9), or a variant thereof. An exemplary HA tag is YPYDVPDYA (SEQ ID NO:10), or a variant thereof.

A tag can be included such as one or more copies of GGGGS (SEQ ID NO: 26), such as 1, 2, 3, 4, 5, 6, 7, 8, 9, or 10, or more, copies. For example, a 6X tag is: GGGGSGGGGSGGGGSGGGGSGGGGGGGGS (SEQ ID NO: 27). A tag can have various functions, including as a linker, for example providing flexibility, and/or for detection of a fusion protein, e.g. using an anti-GGGGS antibody

A flag tag can be included such as DYKDDDDK (SEQ ID NO: 28). A FLAG tag can be used for such functions as to facilitate protein purification, detection, and localization. For example a FLAG tag can be fused to the N- or C-terminus of a protein of interest, allowing for easy identification and isolation of the protein using an anti-FLAG antibody.

An expression construct is provided according to aspects of the present disclosure which includes a nucleic acid encoding a fusion protein including a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein.

The term “expression construct” is used herein to refer to a double-stranded recombinant DNA molecule containing a nucleic acid desired to be expressed and containing appropriate regulatory elements necessary or desirable for the transcription of the operably linked nucleic acid sequence in vitro or in vivo. The term “recombinant” is used to indicate a nucleic acid construct in which two or more nucleic acids are linked and which are not found linked in nature. The term “expressed” refers to transcription of a nucleic acid to produce a corresponding mRNA and/or translation of the mRNA to produce the corresponding protein. Expression constructs can be generated recombinantly or synthetically or by DNA synthesis using well-known methodology.

An expression construct is introduced into a cell using well-known methodology, such as, but not limited to, by introduction of a vector containing the expression construct into the cell. A “vector” is a nucleic acid that transfers an inserted nucleic acid into and/or between host cells becoming self-replicating. The term includes vectors that function primarily for insertion of a nucleic acid into a cell, replication of vectors that function primarily for the replication of nucleic acid, and expression vectors that function for transcription and/or translation of a nucleic acid. Also included are vectors that provide more than one of the above functions.

Vectors include plasmids, viruses, BACs, YACs, and the like. Particular viral vectors illustratively include those derived from adenovirus, adeno-associated virus and lentivirus.

The term “regulatory element” as used herein refers to a nucleotide sequence which controls some aspect of the expression of an operably linked nucleic acid. Exemplary regulatory elements illustratively include an enhancer, an internal ribosome entry site (IRES), an intron; an origin of replication, a polyadenylation signal (pA), a promoter, a transcription termination sequence, and an upstream regulatory domain, which contribute to the replication, transcription, post-transcriptional processing of a nucleic acid. Those of ordinary skill in the art are capable of selecting and using these and other regulatory elements in an expression construct with no more than routine experimentation.

The term “promoter” as used herein refers to a DNA sequence operably linked to a nucleic acid to be transcribed such as a nucleic acid encoding a desired molecule. A promoter is generally positioned upstream of a nucleic acid sequence to be transcribed and provides a site for specific binding by RNA polymerase and other transcription factors.

In addition to a promoter, one or more enhancer sequences may be included such as, but not limited to, cytomegalovirus (CMV) early enhancer element and an SV40 enhancer element. Additional included sequences are an intron sequence such as the beta globin intron or a generic intron, a transcription termination sequence, and an mRNA polyadenylation (pA) sequence such as, but not limited to SV40-pA, beta-globin-pA, the human growth hormone (hGH) pA and SCF-pA. The term “polyA” or “p (A)” or “pA” refers to nucleic acid sequences that signal for transcription termination and mRNA polyadenylation. The polyA sequence is characterized by the hexanucleotide motif AAUAAA. Commonly used polyadenylation signals are the SV40 pA, the human growth hormone (hGH) pA, the beta-actin pA, and beta-globin pA. The sequences can range in length from 32 to 450 bp. Multiple pA signals may be used.

The term “operably linked” as used herein refers to a nucleic acid in functional relationship with a second nucleic acid. The term “operably linked” encompasses functional connection of two or more nucleic acids, such as an oligonucleotide or polynucleotide to be transcribed and a regulatory element such as a promoter or an enhancer element, which allows transcription of the nucleic acid to be transcribed.

An expression construct provided according to aspects of the present disclosure includes a nucleic acid encoding a fusion protein including a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein is or includes a nucleic acid sequence encoding:

(A)
(SEQ ID NO: 4)
MAVSFIGSTENDVGPSQGSYSSTHAMDNLPFVYNTGYNIGYQNAN
VWRISGGFCVGLDGKVDLPVVGSLDGQSIYGLTEEVGLLIWMGDT
NYSRGTAMSGNSWENVFSGWCVGNYVSTQGLSVHVRPVILKRNSS
AQYSVQKTSIGSIRMRPYNGSS

or a variant thereof
   and

(B)
(SEQ ID NO: 11)
IPLLNAKQILGQLDGGSVAVLPVVDSTNQYLLDRIGELKSGDACI
AEYQQAGRGSRGRKWFSPFGANLYLSMFWRLKRGPAAIGLGPVIG
IVMAEALRKLGADKVRVKWPNDLYLQDRKLAGILVELAGITGDAA
QIVIGAGINVAMRRVEESVVNQGWITLQEAGINLDRNTLAAMLIR
ELRAALELFEQEGLAPYLSRWEKLDNFINRPVKLIIGDKEIFGIS
RGIDKQGALLLEQDGVIKPWMGGEISLRSAEK,

or a variant thereof
   wherein the two component proteins are linked directly or through a linker as (A)-(B), and wherein the expression construct optionally further encodes a localization signal peptide and/or exogenous tag in operable linkage with one or both component proteins.

An expression construct provided according to aspects of the present disclosure includes a nucleic acid encoding a fusion protein including a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein is or includes a nucleic acid sequence encoding:

MAVSFIGSTENDVGPSQGSYSSTHAMDNLPFVYNTGYNIGYQNAN
VWRISGGFCVGLDGKVDLPVVGSLDGQSIYGLTEEVGLLIWMGDT
NYSRGTAMSGNSWENVFSGWCVGNYVSTQGLSVHVRPVILKRNSS
AQYSVQKTSIGSIRMRPYNGSSAAATMYPYDVPDYAGYPYDVPDY
AGYPYDVPDYAASIPLLNAKQILGQLDGGSVAVLPVVDSTNQYLL
DRIGELKSGDACIAEYQQAGRGSRGRKWFSPFGANLYLSMFWRLK
RGPAAIGLGPVIGIVMAEALRKLGADKVRVKWPNDLYLQDRKLAG
ILVELAGITGDAAQIVIGAGINVAMRRVEESVVNQGWITLQEAGI
NLDRNTLAAMLIRELRAALELFEQEGLAPYLSRWEKLDNFINRPV
KLIIGDKEIFGISRGIDKQGALLLEQDGVIKPWMGGEISLRSAEK
PKKKRKVDPKKKRKVDPKKKRKV
(SEQ ID NO: 23, nuc-GafD-mTurboID-
HA (nuc-GlycoID) 473 aa),

or a variant thereof.

An expression construct provided according to aspects of the present disclosure includes a nucleic acid encoding a fusion protein including a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein is or includes a nucleic acid sequence encoding:

MAVSFIGSTENDVGPSQGSYSSTHAMDNLPFVYNTGYNIGYQNANVWRI
SGGFCVGLDGKVDLPVVGSLDGQSIYGLTEEVGLLIWMGDTNYSRGTAM
SGNSWENVFSGWCVGNYVSTQGLSVHVRPVILKRNSSAQYSVQKTSIGS
IRMRPYNGSSAAATMGKPIPNPLLGLDSTASIPLLNAKQILGQLDGGSV
AVLPVVDSTNQYLLDRIGELKSGDACIAEYQQAGRGSRGRKWFSPFGAN
LYLSMFWRLKRGPAAIGLGPVIGIVMAEALRKLGADKVRVKWPNDLYLQ
DRKLAGILVELAGITGDAAQIVIGAGINVAMRRVEESVVNQGWITLQEA
GINLDRNTLAAMLIRELRAALELFEQEGLAPYLSRWEKLDNFINRPVKL
IIGDKEIFGISRGIDKQGALLLEQDGVIKPWMGGEISLRSAEKLPPLER
LTL(SEQ ID NO: 22, Cyt-GafD-mTurboID-V₅(cyt-
GlycoID)₄₄₄aa),

or a variant thereof.

An expression construct provided according to aspects of the present disclosure includes a nucleic acid encoding full length GafD (Uniprot: Q47341):

(SEQ ID NO: 3)
MTNFYKVCLAVFILVCCNISHAAVSFIGSTENDVGPSQGSYSSTHAMDN
LPFVYNTGYNIGYQNANVWRISGGFCVGLDGKVDLPVVGSLDGQSIYGL
TEEVGLLIWMGDTNYSRGTAMSGNSWENVFSGWCVGNYVSTQGLSVHVR
PVILKRNSSAQYSVQKTSIGSIRMRPYNGSSAGSVQTTVNFSLNPFTLN
DTVTSCRLLTPSAVNVSLAAISAGQLPSSGDEVVAGTTSLKLQCDAGVT
VWATLTDATTPSNRSDILTLTGASTATGVGLRIYKNTDSTPLKFGPDSP
VKGNENQWQLSTGTETSPSVRLYVKYVNTGEGINPGTVNGISTFTFSYQ,

or a variant thereof.

An expression construct provided according to aspects of the present disclosure includes a nucleic acid encoding a glycan specific binding fragment of GafD lectin which includes the following sequence (amino acids 23-178 of the full-length GafD protein, termed “GafD short” herein:

(SEQ ID NO: 4)
MAVSFIGSTENDVGPSQGSYSSTHAMDNLPFVYNTGYNIGYQNANVWRI
SGGFCVGLDGKVDLPVVGSLDGQSIYGLTEEVGLLIWMGDTNYSRGTAM
SGNSWENVFSGWCVGNYVSTQGLSVHVRPVILKRNSSAQYSVQKTSIGS
IRMRPYNGSS,

or a variant thereof.

A nucleic acid encoding GafD-short is:

(SEQ ID NO: 12)
ATGGCCGTGTCCTTCATCGGCAGCACCGAAAATGATGTGGGCCCTAGCC
AGGGCAGCTACAGCTCTACACACGCCATGGACAACCTGCCTTTCGTGTA
CAACACCGGCTACAATATCGGCTACCAGAACGCCAACGTGTGGCGGATC
TCTGGCGGCTTTTGTGTTGGCCTGGACGGCAAAGTGGATCTGCCTGTTG
TGGGCTCTCTGGACGGCCAGTCTATCTACGGCCTGACAGAGGAAGTGGG
CCTGCTGATCTGGATGGGCGACACCAATTACAGCAGAGGCACAGCCATG
AGCGGCAACAGCTGGGAGAATGTGTTCAGCGGATGGTGCGTGGGCAACT
ACGTGTCCACACAGGGACTGTCTGTGCACGTGCGGCCTGTGATCCTGAA
GAGAAATAGCAGCGCCCAGTACAGCGTGCAGAAAACCAGCATCGGCTCC
ATCAGAATGCGGCCCTACAATGGCAGCTCT.

A nucleic acid encoding miniTurboID is:

(SEQ ID NO: 13)
ATCCCGCTGCTGAACGCTAAACAGATTCTGGGACAGCTGGACGGCGGGA
GCGTGGCAGTCCTGCCTGTGGTCGACTCCACCAATCAGTACCTGCTGGA
TCGAATCGGCGAGCTGAAGAGTGGGGATGCTTGCATTGCAGAATATCAG
CAGGCAGGGAGAGGAAGCAGAGGGAGGAAATGGTTCTCTCCTTTTGGAG
CTAACCTGTACCTGAGTATGTTTTGGCGCCTGAAGCGGGGACCAGCAGC
AATCGGCCTGGGCCCGGTCATCGGAATTGTCATGGCAGAAGCGCTGCGA
AAGCTGGGAGCAGACAAGGTGCGAGTCAAATGGCCCAATGACCTGTATC
TGCAGGATAGAAAGCTGGCAGGCATCCTGGTGGAGCTGGCCGGAATAAC
AGGCGATGCTGCACAGATCGTCATTGGCGCCGGGATTAACGTGGCTATG
AGGCGCGTGGAGGAAAGCGTGGTCAATCAGGGCTGGATCACACTGCAGG
AAGCAGGGATTAACCTGGACAGGAATACTCTGGCCGCTATGCTGATCCG
AGAGCTGCGGGCAGCCCTGGAACTGTTCGAGCAGGAAGGCCTGGCTCCA
TATCTGTCACGGTGGGAGAAGCTGGATAACTTCATCAATAGACCCGTGA
AGCTGATCATTGGGGACAAAGAGATTTTCGGGATTAGCCGGGGGATTGA
TAAACAGGGAGCCCTGCTGCTGGAACAGGACGGAGTTATCAAACCCTGG
ATGGGCGGAGAAATCAGTCTGCGGTCTGCCGAAAAG.

A nucleic acid encoding a NLS is:

(SEQ ID NO: 14)
GACCCCAAGAAGAAGAGGAAGGTGGACCCCAAGAAGAAGAGGAAGGTGG
ACCCCAAGAAGAAGAGGAAGGTG.

A nucleic acid encoding an NES is:

(SEQ ID NO: 15)
CTGCCTCCCCTGGAGCGCCTGACCCTGGAC,

Nucleic acids encoding a linker are:

(SEQ ID NO: 16)
GCGGCCGCCACCATGTACCCGTATGATGTTCCGGATTACGCTGGCTATC
CCTACGACGTGCCCGACTATGCCGGGTACCCCTATGACGTCCCAGACTA
CGCAGCTAGCCTGCAG,
and
(SEQ ID NO: 17)
AAGCTTGCGGCCGCCACCATGGGCAAGCCCATCCCCAACCCCCTGCTGG
GCCTGGACAGCACCGCTAGC

Nucleic acids encoding a protein, or a variant thereof, can be isolated or generated recombinantly or synthetically using well-known methodology.

According to aspects of the present disclosure, contacting the living cell with the fusion protein comprises introducing an expression construct encoding the fusion protein into the cell.

The expression construct may be transfected into cells using well-known methods, such as electroporation, calcium-phosphate precipitation transfection, and lipofection. The cells are screened for presence and/or integration by DNA analysis, such as PCR, Southern blot or sequencing. Cells with the expression construct can be screened for functional expression, for example ELISA or Western blot analysis.

Cells are provided according to aspects of the present disclosure which include the expression construct which includes a nucleic acid encoding a fusion protein including a glycan binding component linked to a mutant E. coli biotin ligase BirA, the glycan binding component capable of specific binding to a glycosylation post-translational modification of a target protein and the mutant E. coli biotin.

Methods of detecting proteins proximal to a target protein according to aspects of the present disclosure include: providing a fusion protein including a glycan binding component linked to a mutant E. coli biotin ligase BirA, the glycan binding component capable of specific binding to a glycosylation post-translational modification of a target protein and the mutant E. coli biotin; contacting the fusion protein with a living cell under compatible biological conditions, whereby the fusion protein specifically binds to a glycosylation post-translational modification of a target protein of the cell; providing biotin to the living cell, whereby the mutant E. coli biotin ligase BirA ligates biotin to proteins proximal to the target protein; and detecting the biotinylated proteins, thereby detecting proteins proximal to the target protein.

The term “compatible biological conditions” refers to conditions which are compatible with living cells and which do not interfere with the desired function and localization of the fusion protein. Physiological conditions is a general term signifying compatible biological conditions and such conditions are well-known.

According to aspects of the present disclosure, detecting the biotinylated proteins includes purifying the biotinylated proteins and detecting the purified biotinylated proteins.

The term “purifying” in the context of purifying the biotinylated proteins for detection refers to separation of the biotinylated proteins from at least one other component present in the system in which the biotinylated proteins were produced. For example, biotinylated proteins are separated from cells in which they are produced, generating purified biotinylated proteins.

According to aspects, the purified biotinylated proteins make up at least about 0.01-100% of the mass, by weight, such as about 0.01%, 0.1%, 1%, 5%, 10%, 25%, 50%, 75%, 85%, 90%, 95%, 96%, 97%, 98%, 99%, or greater than about 99% of the mass, by weight, of material in a sample of purified biotinylated proteins. Such purification is achieved by techniques illustratively including salt, pH, hydrophobic or affinity precipitation, electrophoretic methods such as gel electrophoresis and 2-D gel electrophoresis; chromatography methods such as HPLC, ion exchange chromatography, affinity chromatography, size exclusion chromatography, thin layer and paper chromatography.

According to aspects of the present disclosure, detecting the purified biotinylated proteins includes mass spectrometry.

According to aspects of the present disclosure, mass spectrometry is used in a method for detecting the purified biotinylated proteins. A variety of configurations of mass spectrometers can be used in a method of the present disclosure. In general, a mass spectrometer has the following major components: a sample inlet, an ion source, a mass analyzer, a detector, a vacuum system, and instrument-control system, and a data system. Common mass analyzers include a quadrupole mass filter, ion trap mass analyzer and time-of-flight mass analyzer.

The ion formation process is a starting point for mass spectrum analysis and several ionization methods are available. For example, electrospray ionization (ESI) can be used. Generally described, in ESI a solution containing the material to be analyzed is passed through a fine needle at high potential which creates a strong electrical field resulting in a fine spray of highly charged droplets that is directed into the mass spectrometer. Other ionization procedures include, for example, fast-atom bombardment (FAB) which uses a high-energy beam of neutral atoms to strike a solid sample causing desorption and ionization. Matrix-assisted laser desorption ionization (MALDI) is a method in which a laser pulse is used to strike a sample that has been crystallized in an UV-absorbing compound matrix. Other ionization procedures known in the art include, for example, plasma and glow discharge, plasma desorption ionization, resonance ionization, and secondary ionization.

Electrospray ionization (ESI) has several properties that are useful for methods of assessing an analyte of the present disclosure. For example, the efficiency of ESI can be very high which provides the basis for highly sensitive measurements. Furthermore, ESI produces charged molecules from solution, which is convenient for analyzing analytes and standards that are in solution. In contrast, ionization procedures such as MALDI require crystallization of the material to be analyzed prior to ionization.

Since ESI can produce charged molecules directly from solution, it is compatible with samples from liquid chromatography systems. In liquid chromatography with tandem mass spectrometry (LC-MS-MS), the inlet can be a capillary-column liquid chromatography source. For example, a mass spectrometer can have an inlet for a liquid chromatography system, such as an HPLC, so that fractions flow from the chromatography column into the mass spectrometer. This in-line arrangement of a liquid chromatography system and mass spectrometer is sometimes referred to as LC-MS. An LC-MS system can be used, for example, to separate analytes and standards from complex mixtures before mass spectrometry analysis. In addition, chromatography can be used to remove salts or other buffer components from the sample before mass spectrometry analysis. For example, desalting of a sample using a reversed-phase HPLC column, in-line or off-line, can be used to increase the efficiency of the ionization process and thus improve sensitivity of detection by mass spectrometry.

A variety of mass analyzers are available that can be paired with different ion sources. Different mass analyzers have different advantages as known to one skilled in the art and as described herein. The mass spectrometer and methods chosen for detection depends on the particular assay, for example, a more sensitive mass analyzer can be used when a small amount of ions are generated for detection. Several types of mass analyzers and mass spectrometry methods are described below.

Quadrupole mass spectrometry utilizes a quadrupole mass filter or analyzer. This type of mass analyzer is composed of four rods arranged as two sets of two electrically connected rods. A combination of rf and dc voltages are applied to each pair of rods which produces fields that cause an oscillating movement of the ions as they move from the beginning of the mass filter to the end. The result of these fields is the production of a high-pass mass filter in one pair of rods and a low-pass filter in the other pair of rods. Overlap between the high-pass and low-pass filter leaves a defined m/z that can pass both filters and traverse the length of the quadrupole. This m/z is selected and remains stable in the quadrupole mass filter while all other m/z have unstable trajectories and do not remain in the mass filter. A mass spectrum results by ramping the applied fields such that an increasing m/z is selected to pass through the mass filter and reach the detector. In addition, quadrupoles can also be set up to contain and transmit ions of all m/z by applying a rf-only field. This allows quadrupoles to function as a lens or focusing system in regions of the mass spectrometer where ion transmission is needed without mass filtering. This will be of use in tandem mass spectrometry as described further below.

A quadrupole mass analyzer, as well as the other mass analyzers described herein, can be programmed to analyze a defined m/z or mass range. Since the mass range of analytes and standards will be known prior to an assay, a mass spectrometer can be programmed to transmit ions of the projected correct mass range while excluding ions of a higher or lower mass range. The ability to select a mass range can decrease the background noise in the assay and thus increase the signal-to-noise ratio as well as increasing the specificity of the assay. Therefore, the mass spectrometer can accomplish an inherent separation step as well as detection and identification of analytes and standards.

Ion trap mass spectrometry utilizes an ion trap mass analyzer. In these mass analyzers, fields are applied so that ions of all m/z are initially trapped and oscillate in the mass analyzer. Ions enter the ion trap from the ion source through a focusing device such as an octapole lens system. Ion trapping takes place in the trapping region before excitation and ejection through an electrode to the detector. Mass analysis is accomplished by sequentially applying voltages that increase the amplitude of the oscillations in a way that ejects ions of increasing m/z out of the trap and into the detector. In contrast to quadrupole mass spectrometry, all ions are retained in the fields of the mass analyzer except those with the selected m/z. One advantage to ion traps is that they have very high sensitivity, as long as one is careful to limit the number of ions being tapped at one time. Control of the number of ions can be accomplished by varying the time over which ions are injected into the trap. The mass resolution of ion traps is similar to that of quadrupole mass filters, although ion traps do have low m/z limitations.

Time-of-flight mass spectrometry utilizes a time-of-flight mass analyzer. For this method of m/z analysis, an ion is first given a fixed amount of kinetic energy by acceleration in an electric field (generated by high voltage). Following acceleration, the ion enters a field-free or “drift” region where it travels at a velocity that is inversely proportional to its m/z. Therefore, ions with low m/z travel more rapidly than ions with high m/z. The time required for ions to travel the length of the field-free region is measured and used to calculate the m/z of the ion. One consideration in this type of mass analysis is that the set of ions being studied be introduced into the analyzer at the same time. For example, this type of mass analysis is well suited to ionization techniques like MALDI which produces ions in short well-defined pulses. Another consideration is to control velocity spread produced by ions that have variations in their amounts of kinetic energy. The use of longer flight tubes, ion reflectors, or higher accelerating voltages can help minimize the effects of velocity spread. Time-of-flight mass analyzers have a high level of sensitivity and a wider m/z range than quadrupole or ion trap mass analyzers. Also data can be acquired quickly with this type of mass analyzer because no scanning of the mass analyzer is necessary.

Tandem mass spectrometry can utilize combinations of the mass analyzers described above.

Tandem mass spectrometers can use a first mass analyzer to separate ions according to their m/z in order to isolate an ion of interest for further analysis. The isolated ion of interest is then broken into fragment ions, called collisionally activated dissociation or collisionally induced dissociation, and the fragment ions are analyzed by the second mass analyzer. These types of tandem mass spectrometer systems are called tandem in space systems because the two mass analyzers are separated in space, usually by a collision cell. Tandem mass spectrometer systems also include tandem in time systems where one mass analyzer is used, however the mass analyzer is used sequentially to isolate an ion, induce fragmentation, and then perform mass analysis.

Mass spectrometers in the tandem in space category have more than one mass analyzer. For example, a tandem quadrupole mass spectrometer system can have a first quadrupole mass filter, followed by a collision cell, followed by a second quadrupole mass filter and then the detector. Another arrangement is to use a quadrupole mass filter for the first mass analyzer and a time-of-flight mass analyzer for the second mass analyzer with a collision cell separating the two mass analyzers.

Other tandem systems are known in the art including reflection-time-of-flight, tandem sector and sector-quadrupole mass spectrometry.

Mass spectrometers in the tandem in time category have one mass analyzer that performs different functions at different times. For example, an ion trap mass spectrometer can be used to trap ions of all m/z. A series of rf scan functions are applied which ejects ions of all m/z from the trap except the m/z of ions of interest. After the m/z of interest has been isolated, an rf pulse is applied to produce collisions with gas molecules in the trap to induce fragmentation of the ions. Then the m/z values of the fragmented ions are measured by the mass analyzer. Ion cyclotron resonance instruments, also known as Fourier transform mass spectrometers, are an example of tandem-in-time systems.

Several types of tandem mass spectrometry experiments can be performed by controlling the ions that are selected in each stage of the experiment. The different types of experiments utilize different modes of operation, sometimes called “scans,” of the mass analyzers. In a first example, called a mass spectrum scan, the first mass analyzer and the collision cell transmit all ions for mass analysis into the second mass analyzer. In a second example, called a product ion scan, the ions of interest are mass-selected in the first mass analyzer and then fragmented in the collision cell. The ions formed are then mass analyzed by scanning the second mass analyzer. In a third example, called a precursor ion scan, the first mass analyzer is scanned to sequentially transmit the mass analyzed ions into the collision cell for fragmentation. The second mass analyzer mass-selects the product ion of interest for transmission to the detector. Therefore, the detector signal is the result of all precursor ions that can be fragmented into a common product ion. Other experimental formats include neutral loss scans where a constant mass difference is accounted for in the mass scans. The use of these different tandem mass spectrometry scan procedures can be advantageous when large sets of analytes are measured in a single experiment.

In view of the above, those skilled in the art recognize that different mass spectrometry methods, for example, quadrupole mass spectrometry, ion trap mass spectrometry, time-of-flight mass spectrometry and tandem mass spectrometry, can use various combinations of ion sources and mass analyzers which allows for flexibility in designing customized detection protocols. In addition, mass spectrometers can be programmed to transmit all ions from the ion source into the mass spectrometer either sequentially or at the same time. Furthermore, a mass spectrometer can be programmed to select ions of a particular mass for transmission into the mass spectrometer while blocking other ions. The ability to precisely control the movement of ions in a mass spectrometer allows for greater options in detection protocols which can be advantageous when a large number of analytes are being analyzed.

Different mass spectrometers have different levels of resolution, that is, the ability to resolve peaks between ions closely related in mass. The resolution is defined as R=m/delta m, where m is the ion mass and delta m is the difference in mass between two peaks in a mass spectrum. For example, a mass spectrometer with a resolution of 1000 can resolve an ion with a m/z of 100.0 from an ion with a m/z of 100.1. Those skilled in the art will therefore select a mass spectrometer having a resolution appropriate for the analyte(s) to be detected.

Mass spectrometers can resolve ions with small mass differences and measure the mass of ions with a high degree of accuracy. Therefore, analytes of similar masses can be used together in the same experiment since the mass spectrometer can differentiate the mass of even closely related molecules. The high degree of resolution and mass accuracy achieved using mass spectrometry methods allows the use of large sets of analytes because they can be distinguished from each other.

Mass spectrometry devices and general methods of their use are well known in the art as exemplified in McMaster, M., LC/MS A Practical User's Guide, 2005, John Wiley & Sons, USA; and Hoffmann and Stroobant, Mass Spectrometry Principles and Applications, 2007, John Wiley & Sons, England.

According to aspects of the present disclosure, detecting the purified biotinylated proteins includes chromatography. According to aspects of the present disclosure, detecting the purified biotinylated proteins includes gel electrophoresis. According to aspects of the present disclosure, detecting the purified biotinylated proteins includes gel electrophoresis and transfer of the electrophoresed purified biotinylated proteins to a membrane.

One or more controls or standards can be used to detect biotinylated proteins and/or compare one or more biotinylated proteins obtained under different conditions, e.g. before and after treatment of cells with a test substance.

A test substance may be a natural or synthetic chemical compound, nucleic acid, peptide, protein, saccharide, oligosaccharide, polysaccharide, lipid, or combination of any two or more thereof. Extracts of plants which contain several characterized or uncharacterized components may be a test substance. According to aspects, the test substance is an antisense molecule, an aptamer, siRNA, shRNA, miRNA, a DNAzyme, or a ribozyme.

Embodiments of inventive compositions and methods are illustrated in the following examples. These examples are provided for illustrative purposes and are not considered limitations on the scope of inventive compositions and methods.

### EXAMPLES

### Materials and Methods

Generation of Constructs: The full-length GafD gene was synthesized by ThermoFisher GeneArt from the reported template, described in detail in Saarela, S. et al., The Escherichia coli G-fimbrial lectin protein participates both in fimbrial biogenesis and in recognition of the receptor N-acetyl-D-glucosamine. J Bacteriol 1995, 177 (6), 1477-84. (Uniprot: Q47341), as shown below. The O-GlcNAc binding domain (residues 23-178), which termed “GafD_short,” herein, was specifically chosen for insertion using the primers listed in Table 3.

TABLE 3

| Primer Name | Sequence |
|---|---|
| Hindlll_GafD_ | CCCCCC AAGCTT ATG GCC GTG TCC TTC |
| short_for | ATC GG |
| | (SEQ ID NO: 18) |
| Hindlll_GafD_ | CCCCCC AAGCTT CAG AGC TGC CAT TGT |
| short_rev | AGG G |
| | (SEQ ID NO: 19) |
| Notl_GafD_ | CCCCCC GCGGCCGC AGA GCT GCC ATT GTA |
| short_rev | GGG |
| | (SEQ ID NO: 20) |

The constructs were created via sub-cloning the insert into vectors described in Branon, T. C. et al., Efficient proximity labeling in living cells and organisms with TurboID. Nat Biotechnol 2018, 36 (9), 880-887. V5-miniTurbo-NES_pCDNA3 (Addgene plasmid #107170) and 3xHA-miniTurbo-NLS_pCDNA3 (Addgene plasmid #107172). The target GafD gene was generated through amplification via PCR using MyCycler Thermal Cycler (BioRad) and purified using 1.5% agarose gel (100 V for 40 minutes). The amplified PCR products were purified via QIAquick PCR Purification Kit (Qiagen, 28104). Plasmids were isolated using GeneJet Plasmid Miniprep Kit (ThermoFisher, K0502), using a NanoDrop ND-1000 Spectrometer (ThermoFisher) to confirm their concentrations. The miniTurbo containing plasmids and PCR products were double digested with restriction enzymes, and the plasmid was dephosphorylated using calf intestinal alkaline phosphatase (Quick CIP, NEB: M0525); both were purified using a 0.8% agarose gel and cleaned up via GeneJet Gel Extraction Kit (ThermoFisher, Ko691). The plasmid containing miniTurboID and the GafD gene were ligated using T4 DNA Ligase (NEB, M0202) at 16° C., overnight with shaking (700 rpm). Plasmids were transformed into XL10-Gold Ultracompetent cells (Agilent, 200314). Confirmation of gene insertion was completed using PCR. Cloning was verified by Sanger DNA sequencing. The sequences generated in this study are collected in Table 4. Addgene ID's 184640 (cyt-GlycoID) and 184641 (nuc-GlycoID).

Full length GafD amino acid sequence (Uniprot: Q47341):

(SEQ ID NO: 3)
MTNFYKVCLAVFILVCCNISHAAVSFIGSTENDVGPSQGSYSSTHAMDN
LPFVYNTGYNIGYQNANVWRISGGFCVGLDGKVDLPVVGSLDGQSIYGL
TEEVGLLIWMGDTNYSRGTAMSGNSWENVFSGWCVGNYVSTQGLSVHVR
PVILKRNSSAQYSVQKTSIGSIRMRPYNGSSAGSVQTTVNFSLNPFTLN
DTVTSCRLLTPSAVNVSLAAISAGQLPSSGDEVVAGTTSLKLQCDAGVT
VWATLTDATTPSNRSDILTLTGASTATGVGLRIYKNTDSTPLKFGPDSP
VKGNENQWQLSTGTETSPSVRLYVKYVNTGEGINPGTVNGISTFTFSYQ

Full length DNA sequence encoding GafD

(SEQ ID NO: 21)
ATGACCAACTTCTATAAAGTTTGCCTGGCCGTTTTTATTCTGGTGTGTT
GTAATATTAGCCATGCAGCCGTTAGCTTTATTGGTAGCACCGAAAATGA
TGTTGGTCCGAGCCAGGGTAGCTATAGCAGCACCCATGCAATGGATAAT
CTGCCGTTTGTGTATAACACCGGCTATAACATTGGTTATCAGAATGCAA
ATGTGTGGCGTATTAGCGGTGGTTTTTGTGTTGGTCTGGATGGTAAAGT
TGATCTGCCGGTTGTTGGTAGCCTGGATGGTCAGAGCATTTATGGTCTG
ACCGAAGAAGTGGGTCTGCTGATTTGGATGGGTGATACCAATTATAGCC
GTGGCACCGCAATGAGCGGTAATAGCTGGGAAAATGTTTTTAGCGGTTG
GTGCGTTGGTAATTATGTTAGCACCCAGGGTCTGAGCGTTCATGTTCGT
CCGGTTATTCTGAAACGTAATAGCAGCGCACAGTATAGCGTTCAGAAAA
CCAGCATTGGTAGTATTCGTATGCGTCCGTATAATGGTAGCAGTGCAGG
TAGCGTGCAGACCACCGTGAATTTTAGCCTGAATCCGTTTACACTGAAT
GATACCGTTACCAGCTGTCGTCTGCTGACCCCGAGCGCAGTTAATGTTA
GCCTGGCAGCAATTAGCGCAGGTCAGCTGCCGAGCAGCGGTGATGAAGT
TGTTGCAGGTACAACCAGCCTGAAACTGCAGTGTGATGCGGGTGTTACC
GTTTGGGCAACCCTGACCGATGCAACCACACCGAGCAATCGTAGCGATA
TTCTGACCCTGACAGGTGCAAGCACCGCAACAGGTGTTGGCCTGCGTAT
TTACAAAAATACCGATAGCACACCGCTGAAATTTGGTCCGGATAGTCCG
GTTAAAGGTAATGAAAATCAGTGGCAGCTGAGCACCGGCACCGAAACCA
GTCCGAGCGTTCGTCTGTATGTTAAATATGTGAATACAGGCGAAGGCAT
TAATCCGGGTACAGTTAATGGTATTAGCACCTTTACCTTCAGCTACCAG

TABLE 4
Table 4

| | |
|---|---|
| V5 tag | GKPIPNPLLGLDST |
| | (SEQ ID NO: 9) |
| HA tag | YPYDVPDYA |
| | (SEQ ID NO: 10) |
| GafD-short | MAVSFIGSTENDVGPSQGSYSSTHAMDNLPFVYNT |
| | GYNIGYQNANVWRISGGFCVGLDGKVDLPVVGSLD |
| | GQSIYGLTEEVGLLIWMGDTNYSRGTAMSGNSWEN |
| | VFSGWCVGNYVSTQGLSVHVRPVILKRNSSAQYSV |
| | QKTSIGSIRMRPYNGSS |
| | (SEQ ID NO: 4) |
| miniTurboID | MIPLLNAKQILGQLDGGSVAVLPVVDSTNQYLLDR |
| | IGELKSGDACIAEYQQAGRGSRGRKWFSPFGANLY |
| | LSMFWRLKRGPAAIGLGPVIGIVMAEALRKLGADK |
| | VRVKWPNDLYLQDRKLAGILVELAGITGDAAQIVI |
| | GAGINVAMRRVEESVVNQGWITLQEAGINLDRNTL |
| | AAMLIRELRAALELFEQEGLAPYLSRWEKLDNFIN |
| | RPVKLIIGDKEIFGISRGIDKQGALLLEQDGVIKP |
| | WMGGEISLRSAEK |
| | (SEQ ID NO: 2) |
| Cyt-GafD- | MAVSFIGSTENDVGPSQGSYSSTHAMDNLPFVYNT |
| mTurboID-V₅ | GYNIGYQNANVWRISGGFCVGLDGKVDLPVVGSLD |
| (cyt-GlycoID) | GQSIYGLTEEVGLLIWMGDTNYSRGTAMSGNSWEN |
| | VFSGWCVGNYVSTQGLSVHVRPVILKRNSSAQYSV |
| | QKTSIGSIRMRPYNGSSAAATMGKPIPNPLLGLDS |
| | TASIPLLNAKQILGQLDGGSVAVLPVVDSTNQYLL |
| | DRIGELKSGDACIAEYQQAGRGSRGRKWFSPFGAN |
| | LYLSMFWRLKRGPAAIGLGPVIGIVMAEALRKLGA |
| | DKVRVKWPNDLYLQDRKLAGILVELAGITGDAAQI |
| | VIGAGINVAMRRVEESVVNQGWITLQEAGINLDRN |
| | TLAAMLIRELRAALELFEQEGLAPYLSRWEKLDNF |
| | INRPVKLIIGDKEIFGISRGIDKQGALLLEQDGVI |
| | KPWMGGEISLRSAEKLPPLERLTL |
| | (SEQ ID NO: 22) |
| nuc-GafD- | MAVSFIGSTENDVGPSQGSYSSTHAMDNLPFVYNT |
| mTurboID- | GYNIGYQNANVWRISGGFCVGLDGKVDLPVVGSLD |
| HA(nuc- | GQSIYGLTEEVGLLIWMGDTNYSRGTAMSGNSWEN |
| GlycoID) | VFSGWCVGNYVSTQGLSVHVRPVILKRNSSAQYSV |
| | QKTSIGSIRMRPYNGSSAAATMYPYDVPDYAGYPY |
| | DVPDYAGYPYDVPDYAASIPLLNAKQILGQLDGGS |
| | VAVLPVVDSTNQYLLDRIGELKSGDACIAEYQQAG |
| | RGSRGRKWFSPFGANLYLSMFWRLKRGPAAIGLGP |
| | VIGIVMAEALRKLGADKVRVKWPNDLYLQDRKLAG |
| | ILVELAGITGDAAQIVIGAGINVAMRRVEESVVNQ |
| | GWITLQEAGINLDRNTLAAMLIRELRAALELFEQE |
| | GLAPYLSRWEKLDNFINRPVKLIIGDKEIFGISRG |
| | IDKQGALLLEQDGVIKPWMGGEISLRSAEKPKKKR |
| | KVDPKKKRKVDPKKKRKV |
| | (SEQ ID NO: 23) |
| NES tag | LPPLERLTL |
| | (SEQ ID NO: 8) |
| NLS tag | PKKKRKV |
| | (SEQ ID NO: 7) |

Expression construct encoding Nuc-GlycoID (GafD-short-linker-miniTurboID-NLS)

(SEQ ID NO: 24)
ATGGCCGTGTCCTTCATCGGCAGCACCGAAAATGATGTGGGCCCTAGCC
AGGGCAGCTACAGCTCTACACACGCCATGGACAACCTGCCTTTCGTGTA
CAACACCGGCTACAATATCGGCTACCAGAACGCCAACGTGTGGCGGATC
TCTGGCGGCTTTTGTGTTGGCCTGGACGGCAAAGTGGATCTGCCTGTTG
TGGGCTCTCTGGACGGCCAGTCTATCTACGGCCTGACAGAGGAAGTGGG
CCTGCTGATCTGGATGGGCGACACCAATTACAGCAGAGGCACAGCCATG
AGCGGCAACAGCTGGGAGAATGTGTTCAGCGGATGGTGCGTGGGCAACT
ACGTGTCCACACAGGGACTGTCTGTGCACGTGCGGCCTGTGATCCTGAA
GAGAAATAGCAGCGCCCAGTACAGCGTGCAGAAAACCAGCATCGGCTCC
ATCAGAATGCGGCCCTACAATGGCAGCTCT-GCGGCCGCCACCATGTAC
CCGTATGATGTTCCGGATTACGCTGGCTATCCCTACGACGTGCCCGACT
ATGCCGGGTACCCCTATGACGTCCCAGACTACGCAGCTAGC-ATCCCGC
TGCTGAACGCTAAACAGATTCTGGGACAGCTGGACGGCGGGAGCGTGGC
AGTCCTGCCTGTGGTCGACTCCACCAATCAGTACCTGCTGGATCGAATC
GGCGAGCTGAAGAGTGGGGATGCTTGCATTGCAGAATATCAGCAGGCAG
GGAGAGGAAGCAGAGGGAGGAAATGGTTCTCTCCTTTTGGAGCTAACCT
GTACCTGAGTATGTTTTGGCGCCTGAAGCGGGGACCAGCAGCAATCGGC
CTGGGCCCGGTCATCGGAATTGTCATGGCAGAAGCGCTGCGAAAGCTGG
GAGCAGACAAGGTGCGAGTCAAATGGCCCAATGACCTGTATCTGCAGGA
TAGAAAGCTGGCAGGCATCCTGGTGGAGCTGGCCGGAATAACAGGCGAT
GCTGCACAGATCGTCATTGGCGCCGGGATTAACGTGGCTATGAGGCGCG
TGGAGGAAAGCGTGGTCAATCAGGGCTGGATCACACTGCAGGAAGCAGG
GATTAACCTGGACAGGAATACTCTGGCCGCTATGCTGATCCGAGAGCTG
CGGGCAGCCCTGGAACTGTTCGAGCAGGAAGGCCTGGCTCCATATCTGT
CACGGTGGGAGAAGCTGGATAACTTCATCAATAGACCCGTGAAGCTGAT
CATTGGGGACAAAGAGATTTTCGGGATTAGCCGGGGGATTGATAAACAG
GGAGCCCTGCTGCTGGAACAGGACGGAGTTATCAAACCCTGGATGGGCG
GAGAAATCAGTCTGCGGTCTGCCGAAAAG-GAATTCAGCAGGGCC-GAC
CCCAAGAAGAAGAGGAAGGTGGACCCCAAGAAGAAGAGGAAGGTGGACC
CCAAGAAGAAGAGGAAGGTG-TGA

Expression construct encoding Cyt-GlycoID (GafD-short-linker miniTurboID-NES)

(SEQ ID NO: 25)
ATGGCCGTGTCCTTCATCGGCAGCACCGAAAATGATGTGGGCCCTAGCC
AGGGCAGCTACAGCTCTACACACGCCATGGACAACCTGCCTTTCGTGTA
CAACACCGGCTACAATATCGGCTACCAGAACGCCAACGTGTGGCGGATC
TCTGGCGGCTTTTGTGTTGGCCTGGACGGCAAAGTGGATCTGCCTGTTG
TGGGCTCTCTGGACGGCCAGTCTATCTACGGCCTGACAGAGGAAGTGGG
CCTGCTGATCTGGATGGGCGACACCAATTACAGCAGAGGCACAGCCATG
AGCGGCAACAGCTGGGAGAATGTGTTCAGCGGATGGTGCGTGGGCAACT
ACGTGTCCACACAGGGACTGTCTGTGCACGTGCGGCCTGTGATCCTGAA
GAGAAATAGCAGCGCCCAGTACAGCGTGCAGAAAACCAGCATCGGCTCC
ATCAGAATGCGGCCCTACAATGGCAGCTCT-AAGCTTGCGGCCGCCACC
ATGGGCAAGCCCATCCCCAACCCCCTGCTGGGCCTGGACAGCACCGCTA
GC-ATCCCGCTGCTGAACGCTAAACAGATTCTGGGACAGCTGGACGGCG
GGAGCGTGGCAGTCCTGCCTGTGGTCGACTCCACCAATCAGTACCTGCT
GGATCGAATCGGCGAGCTGAAGAGTGGGGATGCTTGCATTGCAGAATAT
CAGCAGGCAGGGAGAGGAAGCAGAGGGAGGAAATGGTTCTCTCCTTTTG
GAGCTAACCTGTACCTGAGTATGTTTTGGCGCCTGAAGCGGGGACCAGC
AGCAATCGGCCTGGGCCCGGTCATCGGAATTGTCATGGCAGAAGCGCTG
CGAAAGCTGGGAGCAGACAAGGTGCGAGTCAAATGGCCCAATGACCTGT
ATCTGCAGGATAGAAAGCTGGCAGGCATCCTGGTGGAGCTGGCCGGAAT
AACAGGCGATGCTGCACAGATCGTCATTGGCGCCGGGATTAACGTGGCT
ATGAGGCGCGTGGAGGAAAGCGTGGTCAATCAGGGCTGGATCACACTGC
AGGAAGCAGGGATTAACCTGGACAGGAATACTCTGGCCGCTATGCTGAT
CCGAGAGCTGCGGGCAGCCCTGGAACTGTTCGAGCAGGAAGGCCTGGCT
CCATATCTGTCACGGTGGGAGAAGCTGGATAACTTCATCAATAGACCCG
TGAAGCTGATCATTGGGGACAAAGAGATTTTCGGGATTAGCCGGGGGAT
TGATAAACAGGGAGCCCTGCTGCTGGAACAGGACGGAGTTATCAAACCC
TGGATGGGCGGAGAAATCAGTCTGCGGTCTGCCGAAAAG-CTGCAG-CT
GCCTCCCCTGGAGCGCCTGACCCTGGAC-TAA

Mammalian Cell Culture and Transfection: Cells were obtained from ATCC. All consumables (pipette tips, glass Pasteur pipettes, Eppendorf tubes) were sterilized via autoclave. HeLa cells were cultured in DMEM (Sigma Aldrich, D6429) supplemented with

10% (v/v) HyClone Fetal Bovine Serum (Cytiva, SH30396.03) and 1% HyClone Penicillin-Streptomycin solution (Cytiva, SV30010) at 37° C. under 5% CO2. All mammalian cell manipulations were done inside a laminar flow hood sterilized with 70% ethanol. To seed the plates, the cells were carefully washed with sterile 10 mL of PBS pH 7.4 (1X) (ThermoFisher, 10010-023). 1.5 mL of Trypsin 0.25% (1X) solution (Cytiva, SV30031.01) was added to the flask and incubated for 3-5 minutes at 37° C. under 5% CO2. The trypsin was then neutralized with serum-containing growth media, where the trypsin can be further removed via centrifugation (300×g for 3 minutes) in a sterile 15 mL centrifuge tube (FisherScientific, 14-955-237). The cell pellet was washed with PBS and recentrifuged. The cells can then be seeded into desired flask (2.1×10⁶ cells for T-75 flask [USAScientific, CC7682-4875], 2.2×10⁶ cells for 100 mm dish [FisherScientific, FB0875713], 0.3×10⁶ cells for 6-well dish [FisherScientific, 07-200-83], 0.1×10⁶ cells for 12-well dish [Corning, 3512]). According to the manufacturer's protocol, cells were transfected using TransIT-LT1 transfection reagent (Mirus Bio LLC, MIR 2304) for transient transfection. The transfected cells were incubated for 48-72 hours before use. General reagents used in this study are collected in Table 5.

TABLE 5

| Culture Vessel | 24-well | | 12-well | | 6-well | | 100 mm | |
|---|---|---|---|---|---|---|---|---|
| Surface Area | 1.9 | cm² | 3.8 | cm² | 9.6 | cm² | 59 | cm² |
| Complete | 0.5 | mL | 1.0 | mL | 2.5 | mL | 15.5 | mL |
| Growth Media | | | | | | | | |
| Serum-free Media | 50 | μL | 100 | μL | 250 | μL | 1.5 | mL |
| DNA (1 μg/μL) | 0.5 | μL | 1 | μL | 2.5 | μL | 15 | μL |
| TransIT-LT1 | 1.5 | μL | 3 | μL | 7.5 | μL | 45 | μL |
| Reagent | | | | | | | | |

Western Blot: To analyze cell lysates via immunoblot, cells were collected with a cell scraper from plates in RIPA buffer containing protease inhibitors (Thermo 89900 and Roche 11873580001). Cell lysates were briefly sonicated and centrifuged (12,000×g for 10 minutes at 4° C.) to collect the soluble protein fraction. Protein concentration was determined via Pierce Rapid Gold BCA Protein Assay Kit (ThermoFisher, A53225). Samples were boiled in SDS gel loading buffer for 5 minutes. Proteins were separated on a 4-12% gradient gel (NuPAGE™ 4 to 12%, Bis-Tris, 1.0-1.5 mm, Mini Protein Gels; ThermoFisher, NP0321BOX) and transferred to a nitrocellulose membrane (iBlot™ 2 Transfer Stacks, nitrocellulose, mini; ThermoFisher, IB23002) using an iBlot 2 dry blotting system (ThermoFisher, IB21001). After blocking with 5% w/v bovine serum albumin (Research Products International, A30075) in PBST buffer (PBS+0.4% Triton X-100) for 1 hour, the membrane was incubated with the appropriate antibody following the manufacture's protocol. The signals from the antibodies were detected via the iBright™ FL1500 instrument (ThermoFisher, A44115). The membranes were incubated with Cy5- or HRP-conjugated streptavidin to detect biotinylated proteins. All antibodies used in this study are collected in Table 6.

TABLE 6

| Target | Conjugate | Host | Supplier | Dilution |
|---|---|---|---|---|
| Biotin | HRP | Goat | CST - 7075S | 1:1000-1:3000 |
| Biotin | Cy5 | Bacterial | Invitrogen - | 1:1000 |
| | | | SA1011 | |
| Flag | N/A | Mouse | Sigma - F3165 | 1:1000 |
| HA | N/A | Mouse | BioLegend - | 1:1000 |
| | | | 901503 | |
| HA | N/A | Rabbit | CST - 3724S | 1:1000 |
| Mouse IgG | HRP | Goat | Thermo - | 1:10000-1:200000 |
| | | | A28175 | |
| OGT | N/A | Rabbit | CST - 24083S | 1:1000 |
| Rabbit IgG | HRP | Donkey | Thermo - | 1:10000-1:200000 |
| | | | 31458 | |
| V5 | N/A | Rabbit | CST - 13202S | 1:1000 |
| V5 | N/A | Rabbit | Thermo - | 1:1000-1:5000 |
| | | | PA1-993 | |
| Rabbit IgG | Alexa | Goat | A27039 | 1:1000 |
| | Fluor 555 | | | |
| Rabbit IgG | Alexa | Goat | A-11008 | 1:1000 |
| | Fluor 488 | | | |
| O-GlcNAc | N/A | Rabbit | CST - 82332S | 1:1000 |
| MultiMab | | | | |

Immunofluorescence of GlycoID constructs: HeLa cells were seeded onto 8-well glass chamber slides (Thermo #154534PK) before transfection with mTurbo or GlycoID constructs. After transfection and expression for 48 h, the media was removed and the cells were fixed with 4% paraformaldehyde, then washed three times with PBS. Cells were permeabilized with 0.3% Triton X-100 in PBS, then washed, and blocked with 10% goat serum overnight at 4 C. Blocking agent was washed twice with PBS, then rabbit primary antibody to the HA- or V5-epitope was added at 1:500 dilution in 1.5% goat serum. After probing overnight at 4° C., the cells were washed with PBS three times (5 min per wash) and probed with anti-rabbit AF555 secondary antibody (1:1000) in 1.5% goat serum. Cells were given four final 5-minute washes before mounting with DAPI-containing mountant (Thermo #P36966). Fluorescent images were captured on a Carl Zeiss AX10 microscope and processed using the Carl Zeiss Zen 2 software.

Biotin Labeling with Fusion Constructs: For biotin labeling experiments of transiently transfected cells, biotin was added 48-72 hours after transfection. Biotin (Carbosynth, 58-85-5) was diluted to 1 mM (or desired concentration) in serum-free growth media and added directly to cells at the indicated final concentrations. The cells were incubated at 37° C. for the desired amount of time. For western blots and proteomics, labeling was stopped by washing with cold PBS and freezing at −80° C.

siRNA Knockdown: For OGT knockdown, cells were transfected with Dharmacon™ ON-TARGET plus SMART pool human OGT siRNA (#L-019111-00-0005), SMART pool human OGA siRNA (#L-012805-00-0005), or ON-TARGET control pool non-targeting pool siRNA (#D001810 Oct. 5) as control using DharmaFECT transfection reagent, as described by the manufacturer. The SMART pool consists of a combination of 4 different siRNA oligomers optimized for a knockdown in human cell lines, which were used here instead of two distinct siRNA sequences for OGT knockdown and its corresponding SMART pool control knockdown. The dried siRNA pellets were recentrifuged and resuspended in RNase-free 1x siRNA buffer (60 mM KCl, 6 mM HEPES-pH 7.5, and 0.2 mM MgCl2) to a final concentration of 20 UM and aliquoted into 20 μL samples. Plates were seeded to the desired confluency. For transfection, the siRNA aliquot was diluted to 5 μM and transfected using the amounts found in Table 7.

TABLE 7

| | | Tube 1: diluted | | Tube 2: diluted | | | |
|---|---|---|---|---|---|---|---|
| | | siRNA | | DharmaFECT | | | |
| | | Volume | | Volume of | | | Total |
| | | of 5 μM | Serum-free | DharmaFECT | Serum-free | Complete | transfection |
| Plating | Surface Area | siRNA | media | reagent | media | media | volume |
| Format | (cm²/well) | (μL) | (μL) | (μL) | (μL) | (μL/well) | (μL/well) |
| 96 | 0.3 | 0.5 | 9.5 | 0.05-0.5 | 9.95-9.5 | 80 | 100 |
| 24 | 2 | 2.5 | 47.5 | 0.25-2.5 | 49.75-47.5 | 400 | 500 |
| 12 | 4 | 5 | 95 | 0.5-5.0 | 99.5-95.0 | 800 | 1000 |
| 6 | 10 | 10 | 190 | 1.0-10.0 | 199.0-190.0 | 1600 | 2000 |

For 24-well plates, 2 μL of DharmaFECT reagent was sufficient for this siRNA transfection. The reagents were gently mixed via pipetting and incubated for 5 minutes at room temperature. The tubes were then combined and incubated for an additional 20 minutes. The media was removed from the plate and replaced with the appropriate amount of transfection reagent. For protein analysis, the transfected plates were incubated at 37 0C under 5% CO2 for 48-96 hours.

Sample Preparation for Proteomics: Cells were cultured in d-100 mm TC-treated Petri dishes. All cells were transiently expressing the desired construct. All cells were labeled with 100 UM biotin using the aforementioned methods. Labeling was stopped by washing with cold PBS and freezing at −80 oC. The cells were detached from the plate via scrapper with 2% SDS-containing RIPA lysis buffer (150 mM NaCl, 0.5 mM tris, 1% NP40, 2.0% SDS) and collected in Eppendorf tubes. The cells were lysed via passage through a needle (at least 10 passes) or sonication and clarified with centrifugation at 10,000×g for 10 minutes at 4° C.

To enrich biotinylated proteins, 100 μL of streptavidin-coated magnetic beads (NEB S1410S) were washed twice with RIPA buffer and then incubated with clarified lysates

(400 μg protein) with rotation at 4° C. overnight. The magnetic beads were then washed once with 500 μL RIPA buffer, once with 500 μL wash buffer (50 mM Tris, pH 7.4, 2% SDS), and twice with 500 μL RIPA buffer. Magnetic beads were resuspended in 500 μL 10 mM DTT (Dithiothreitol, GoldBio 27565-41-9) in PBS at 37° C. for 30 minutes, which was then cooled to room temperature. The supernatant was discarded. The magnetic beads were then resuspended in 1 mL 30 mM iodoacetamide (Sigma, 16125) (protect from light) at r.t. for 30 minutes. The supernatant was discarded, and the beads were washed with pure mass-spec grade water. The magnetic beads were resuspended in 300 μL 50% MeCN/50% water (Fisher Chemical, 75-05-8; Fisher Chemical, 7732-18-5) (ms-grade). The proteins were then digested with Lys-C protease (Thermo Scientific, 90051) with a 1:100 ratio Lys-C to protein sample (˜0.3 μL for 50 μL resin) at 37° C. for 16 hours without shaking. The proteins were further digested with SOLu-Trypsin (Sigma, EMS0004) at a ratio of 1:20 trypsin weight to sample weight (50 μL resin, 3 μL Trypsin) at 47° C. for one hour, then cooled to 37° C. for four hours with rotation. The digestion was quenched by bringing the mixture to a final concentration of 1% formic acid (Thermo Scientific, 85178). The beads were removed from the mixture via magnet (or centrifugation) and washed twice with 200 μL 50% MeCN and once M. S.-water. Bead fragments were removed with centrifugation (10,000×g for 10 minutes). Samples were concentrated via speed vac set to 40° C., and the residues were stored at −80° C. Following the manufacturer's protocol, peptide concentrations were determined via Pierce™ Quantitative Fluorometric Peptide Assay kit (ThermoFisher, 23290). Detergents were detected using an SDS assay, using Stains-all dye. A stock solution of 1.8 mM stains-all was made using 50% propanol: water (protect from light) (e.g., 10 mL solution needs 10 mg of stains-all). A 90 μM working solution was diluted from the stock solution in 5% formamide (OmniPur, 75-12-7) (e.g. for 5 mL; mix 0.25 mL stock, 0.25 mL formamide, 4.5 mL water, 2.5% propanol final). This solution can be stored at room temperature for ˜four days in the dark. Pipette 1 μL of sample and 1 μL of a standard curve SDS (FisherScientific, BP166-500) sample (0.02-0.1%) into a 96 well plate. Standard curve used started at 0.02% with increments of 0.01% (e.g. 0.02, 0.03, 0.04, etc.). 200 UL of the working solution was added into each well with a sample or standard (keep the plate protected from the light). The plate was read using a plate reader at 445 nm. The samples should have a minimum amount of SDS to prevent damage to the mass spectrum column.

Proteomics: All mass spectra were analyzed with MaxQuant software version 1.6.10.43. MS/MS spectra were searched against the Homo Sapiens Uniprot protein sequence database based on version Jun. 16, 2021. Carbamidomethylation of cysteines was searched for as a fixed modification. Oxidation of methionines and acetylation of protein N-terminal and O-GlcNAc proteins termed as HexNac (ST) in MaxQuant software were searched against as variable modification. The enzyme was set to trypsin and Lys-C in a specific mode. All other parameters were used as default in MaxQuant. Label-free quantification was selected for group-specific parameters. Using Perseus, all contaminates identified by MaxQuant (streptavidin, reversed proteins, peptides with sequences <=2, etc.) are filtered out. Then the data is categorically grouped, using data with at least 3 out of 4 positive replicates to do a T-Test and plot in a Volcano plot. Note: the serum-starved cyt-GlycoID (30 min of labeling) only had two successful proteomics runs, so its positive hits were 2 out of 2 replicates.

### Data Analysis

For each condition, protein hits that were exclusive to each condition were combined with the significantly identified proteins identified by Volcano plot analysis. These comprehensive lists for each condition were cross-referenced with the dataset from the O-GlcNAcome website as published in Wulff-Fuentes, E. et al., The human O-GlcNAcome database and meta-analysis. Scientific Data 2021, 8 (1), 25. Proteins were also analyzed against the OGT Protein Interaction Network (OGT-PIN) downloaded from the OGT-PIN website as published in Ma, J. et al., OGT Protein Interaction Network (OGT-PIN): A Curated Database of Experimentally Identified Interaction Proteins of OGT. Int J Mol Sci 2021, 22 (17). Total interactome analysis was performed using the STRING online protein-protein association network database with the following parameter settings:
Input
Multiple proteins
Organism: Homo sapiens
Settings
Network type: Physical Subnetwork
Meaning of network edges: evidence
Active interaction sources: Experiments only (“textmining” and “databases” were de-selected)
Minimum required interaction score: medium confidence (0.400)
Max number of interactors to show: 1st shell: query proteins only/2nd shell: none Clusters
Clustering options: kmeans clustering
Number of clusters: varied until significance was not met; 0-6 clusters were identified for each condition.
Significance: p≤0.05

### In Vitro Proximity Labeling of Live Cells.

HeLa or HEK293T cells were seeded on sterile plates and transfected with expression constructs encoding cyt-mTurbo, nuc-mTurbo, cyt-GlycoID, or nuc-GlycoID with at least 48 hours of incubation in DMEM at 37° C. Media were then replaced with media supplemented with 100 μM biotin and allowed to incubate for 6 hours (or alternative times/concentrations as indicated). Then, cells were rinsed with phosphate-buffered saline (PBS) twice before freezing. Cells were harvested via scraping cells off the plates with RIPA buffer and lysed via a passage through a needle (at least 10 passes). For siRNA K_{D}, cells were first transfected with expression constructs encoding GlycoID fusion proteins, followed by 24-48 hours of expression before transfection with Dharmacon ON-TARGET plus SMART pool human OGT siRNA, SMART pool human OGA siRNA, or ON-TARGET scrambled control (nontargeting pool) siRNA 24 hours before biotin induction. Plasmids generated in this research are available via the Addgene repository with ID #184640 (cyt-GafD-mTurboID-V5) and ID #184641 (nuc-GafD-mTurboID-HA).

### Immunofluorescence Staining.

HeLa cells were seeded on eight-well glass chamber slides and transfected with TurboID or GlycoID plasmids. After 48 hours of expression, cells were fixed, permeabilized, and probed for epitope tags: V5 for cytosolic constructs or HA for nuclear constructs. Anti-rabbit Alexa-Fluor555 secondary antibodies and DAPI nuclear staining were used to display localization.

### Preparation of Samples for Proteomics

Following biotin labeling, cells were harvested with 100 μL of RIPA buffer (150 mM NaCl, 0.5 mM tris, 1% NP40, 0.1% SDS) and lysed via a passage through a needle (at least 10 passes). To enrich biotinylated proteins, samples containing 400 μg of total protein were incubated with streptavidin-coated magnetic beads overnight at 4° C. The beads were then washed with RIPA buffer, wash buffer (50 mM Tris, pH 7.4, 2% SDS), and twice more with RIPA buffer. The beads were then resuspended in DTT in PBS and treated with iodoacetamide. The beads were then washed with MS-grade water and were resuspended in MS-grade 50% MeCN/50% water. The samples were digested with Lys-C protease for 16 h and with SOLu-Trypsin for 1 hours (47° C.) and then for 4 hours (37° C.). The supernatants were quenched with formic acid, and the magnetic beads were removed. Samples were dried via vacuum centrifugation and stored at −80° C.

### Proteomic Liquid Chromatography-Tandem Mass Spectrometry Analysis.

Samples were solubilized in 1% trifluoroacetic acid. An EASY nLC UPLC system was used to elute peptides onto a Fusion Tribrid mass spectrometer (Thermo Scientific). MS1 profiling was performed in a 375-1600 m/z range at a resolution of 70,000. MS2 fragmentation was carried out on the top 15 ions by using a 1.6 m/z window and a normalized collision energy of 29 using higher-energy collision-induced dissociation with a dynamic exclusion of 15 s.

### Proteomic Data Analysis

All mass spectra were analyzed with MaxQuant software version 1.6.10.43. MS/MS spectra were searched against the Homo Sapiens Uniprot protein sequence database based on version Jun. 16, 2021. Carbamidomethylation of cysteines was searched for as a fixed modification. Oxidation of methionines and acetylation of protein N-terminal and O-GlcNAc proteins termed as HexNac (ST) in MaxQuant software were searched against as variable modification. The digestion enzyme was set to trypsin and LysC in a specific mode. All other parameters were used as default in MaxQuant. Label-free quantification was selected for group-specific parameters. Using Perseus, all contaminates identified by MaxQuant (streptavidin, reversed proteins, peptides with sequences≤2, etc.) are filtered out. Then, the data was categorically grouped, peptides filtered for presence in at least three out of four positive replicates, analyzed by t-test analysis, and plotted in a Volcano plot. Note: the serum-starved cyt-GlycoID (30 min of labeling) only had two successful proteomic runs, so for this condition, positive hits were assigned when peptides were present in both replicates. Hits were scored by t-test analysis (p<0.05) and fold-change (log 2>+0.05). The MS proteomic data have been deposited to the ProteomeXchange Consortium via the PRIDE partner repository with the data set identifiers PXD033026, PXD033043, PXD033044, PXD033062, PXD033063, and PXD033066.

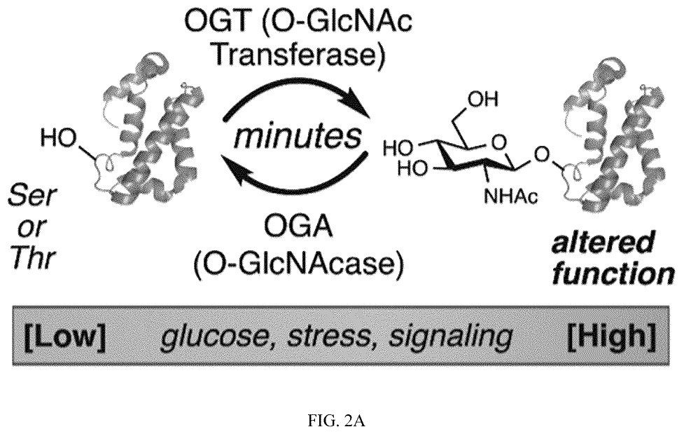

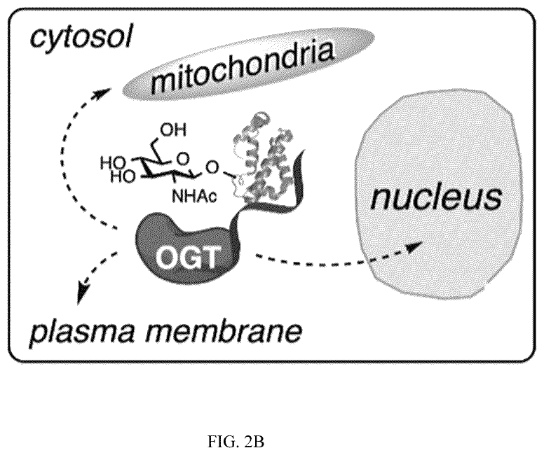

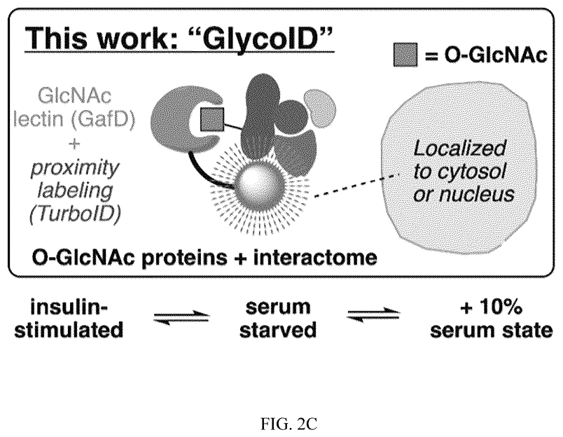

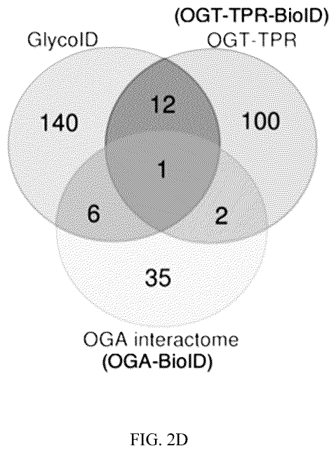

Compositions and methods according to aspects of the present disclosure provided detection and characterization of functional O-GlcNAc “activity hubs” that responded to conditions in live cells, including insulin stimulation and serum feeding (FIG. 2C).

Spatial control within cells was achieved using constructs according to aspects of the present disclosure targeted to the nucleus or cytoplasm, revealing location-specific O-GlcNAc hubs. Rapid labeling conditions within 30 min of signal induction confirmed the possibility of tracking O-GlcNAc modifications in short timescales relevant to signal transduction. Further, compositions and methods according to aspects of the present disclosure provide intracellular O-GlcNAc labeling of functional protein hubs during insulin signaling and growth serum nutrient sensing.

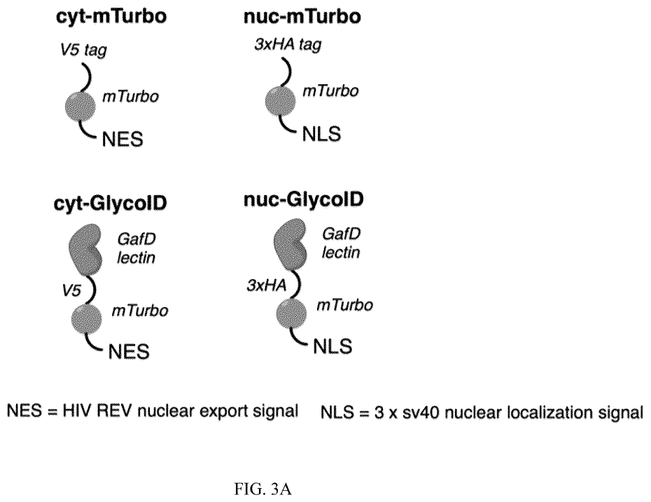

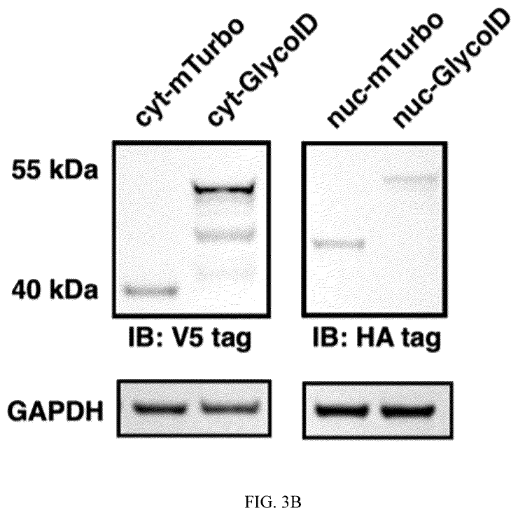

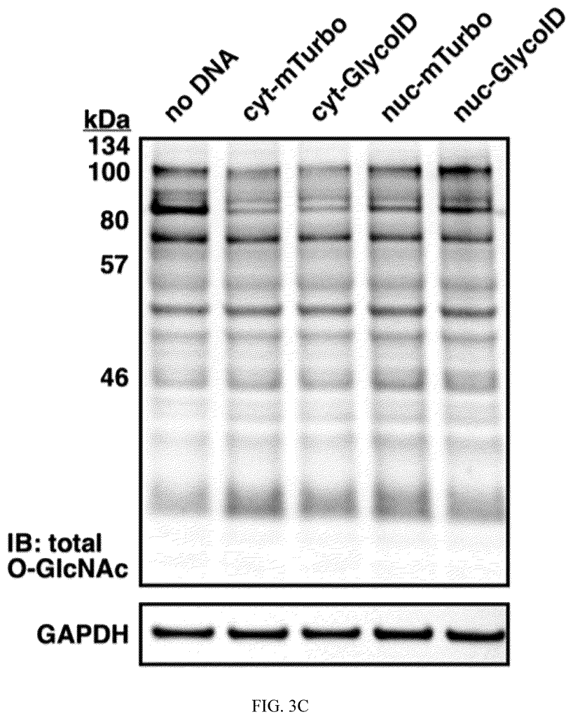

In this example, the term “GlycoID” is used to denote a fusion protein including a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein, wherein the glycan binding component is GafD lectin and the mutant E. coli biotin ligase BirA is miniTurboID (mTurbo) of SEQ ID NO:2, see FIG. 3A. The term “nuc-GlycoID” is used to denote a GlycoID fusion protein which includes a nuclear localization signal and “cyt-GlycoID” is used to denote a GlycoID fusion protein which includes a cytoplasm localization signal. The terms “cyt-mTurbo” and “nuc-mTurbo” are used to denote mTurbo with a cytoplasm localization signal and mTurbo with a nuclear localization signal, respectively.

miniTurboID (mTurbo) uses the nontoxic substrate biotin to attach nonhydrolyzable biotin tags to proteins within a small, ca. <10 nm radius from a bound target protein. The relatively modest 28 kDa size of mTurbo enables efficient expression in human cell lines when fused to protein-targeting domains, see Branon TC et al., Nat. Biotechnol 2018, 36, 880-887. Furthermore, since the parent biotin ligase BirA requires substantial biotin for labeling (KM ˜ 5 μM),²⁸ cellular labeling can be conducted in media that lack biotin, including Dulbecco's modified Eagle's media (DMEM).

GafD (10io) described in detail in Merckel MC et al., J. Mol. Biol 2003, 331, 897-905 and BirA (3rux) described in detail in Duckworth BP et al., Chem. Biol 2011, 18, 1432-1441 were used in fusion protein constructs according to aspects of the present disclosure bearing an N-terminal GafD, a short linker containing an HA or V5 tag, such as for immunoblotting and immunofluorescence, and the C-terminal mTurbo domain containing a localization sequence. Sequences are shown in Table 4.

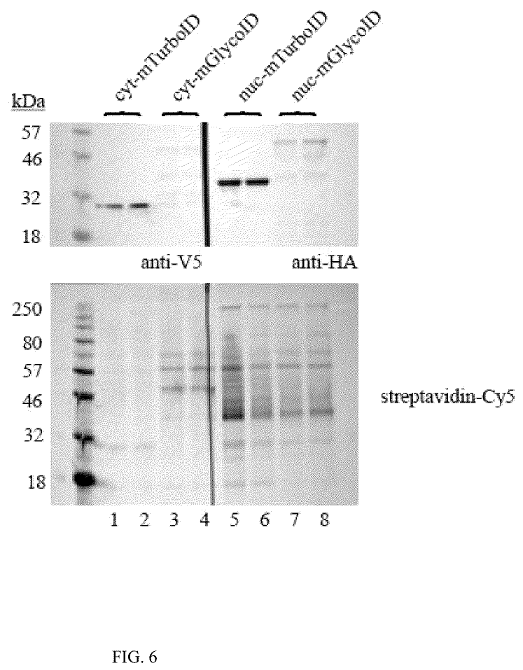

O-GlcNAcylation primarily occurs in the nucleus and cytoplasm of cells. Two GlycoID constructs were generated according to aspects of the present disclosure that localized to these subcellular compartments, cyt-GlycoID and nuc-GlycoID, see Table 4 and FIG. 3A. As control constructs that were not directed to GlcNAc-modified proteins, cyt-mTurbo and nuc-mTurbo were expressed that lacked the GafD domain but maintained cytosolic or nuclear expression patterns, as judged by immunofluorescence staining against the V5- and 3xHA-expression tags. These four constructs were transiently expressed in HeLa, see FIG. 3B, and HEK293T cells, see FIG. 6. FIG. 6 shows a Western blot for the expression of cyt-mTurboID (1, 2), cyt-GlycoID (3, 4), nuc-mTurboID (5, 6), nuc-GlycoID (7, 8) in HEK293T cells. All experiments used 100 μM biotin and were allowed to label for 6 hours. Expected sizes: cyt-mTurbo, 31.2 kDa; cyt-GlycoID, 48.6 kDa; nuc-mTurbo, 35.3 kDa; nuc-GlycoID, 52.5 kDa.

In some experiments, biotin induction was at 500 μM, 0.5 hours reaction, followed by cell lysis and total cell blotting. Dual-fluorescence blots were probed with a pan-O-GlcNAc antibody (O-GlcNAc MultiMAb) and visualized with an AlexaFluor-555 secondary and streptavidin-Cyanines fluorescent conjugates.

In some experiments, the activity of these constructs to biotinylate intracellular proteins was shown by incubating GlycoID-expressing cells with 100 μM biotin for 6 h, followed by cell lysis and immunoblotting for O-GlcNAcylation and biotinylation. Verifying O-GlcNAc binding and labeling activity was shown by dual imaging with anti-O-GlcNAc/AF555, overlaid with streptavidin/Cy5. Overlapping signals bands demonstrated on-target GlycoID labeling of O-GlcNAcylated proteins in HeLa cells, with more substantial overlap observed in the cyt-GlycoID system. Some differences in O-GlcNAc staining were demonstrated. To verify that overexpression of GlcNAc-binding constructs did not disrupt O-GlcNAc functions and affect cellular O-GlcNAc levels, a global O-GlcNAc immunoblotting experiment was performed in cells exposed to standard GlycoID labeling conditions, see FIG. 3C. Minimal disruptions were observed between the four proximity labeling constructs using the O-GlcNAc MultiMAb pool of antibodies raised against nonspecific O-GlcNAc epitopes and traditional chemiluminescence detection, which gives a stronger signal than the fluorescent blotting system.

### Optimization and Validation of Intracellular O-GlcNAc Labeling

The labeling activity of GlycoID constructs was characterized following transient expression in Hela cells. Concentration variations (0 μM, 25 μM, 50 μM, 100 μM, 250 μM, and 500 μM) and time course (10 min, 1 hour, 6 hours) for the nuc-GlycoID (GafD-mTurbo-NLS) and cyt-GlycoID (GafD-mTurbo-NES) constructs were used.

Biotinylated proteins were visualized using a streptavidin-Cy5 fluorescent conjugate. O-GlcNAc engineering with RNA silencing of OGT or OGA (siOGT or siOGA, respectively), was performed followed by GlycoID labeling with biotin. Knockdowns were initiated after 48 hours of GlycoID expression and 24 hours before labeling with biotin using a Horizon Discovery “SMART pool” scramble control, OGT knockdown pool, or OGA knockdown pool as a combination of four siRNA oligonucleotides per target. Immunoblots for HA tag, V5 tag, and GAPDH were performed as controls for each experiment.

To initiate proximity labeling, GlycoID constructs were expressed in Hela cells for 48h. Treatment of biotin in a range from 25 to 500 μM concentrations was applied from an aqueous stock, and labeling reactions were allowed to proceed from 10 min to 6h. Significant dose- and time-dependent labeling of soluble nucleocytoplasmic proteins were observed in Hela cells. Balancing concentration and time identified detectable labeling as soon as 10 min at 500 μM [biotin], compared to maximal labeling, which peaked under all concentrations at 6 h. Overnight labeling did not lead to more robust labeling.

Overall O-GlcNAc levels were engineered in cells to confirm specificity for GlcNAc-driven protein labeling. The constructs' partial validation of O-GlcNAc binding came from control experiments where either OGT or OGA, the two enzymes that add and remove O-GlcNAc, was suppressed before nuc-GlycoID labeling was labeled induced with biotin treatment. It was hypothesized that RNA silencing (siRNA) directed to endogenous OGT or OGA would affect O-GlcNAc levels but not the expression of GlycoID, leading to altered labeling. It was found that knockdown of OGT led to a suppression of OGT protein levels.

However, the number of biotin-labeled protein bands after 24 hours of siRNA-OGT (siOGT) treatment was not significantly perturbed for either construct. On the other hand, knockdown of OGA (siOGA) was expected to elevate both O-GlcNAc levels and labeling. Slightly elevated O-GlcNAcylation levels and the intensity of the biotin bands versus scrambled siRNA controlwere observed. The relatively weak intensity of the dual-fluorescence blotting was a limitation for definitive effect tracking.

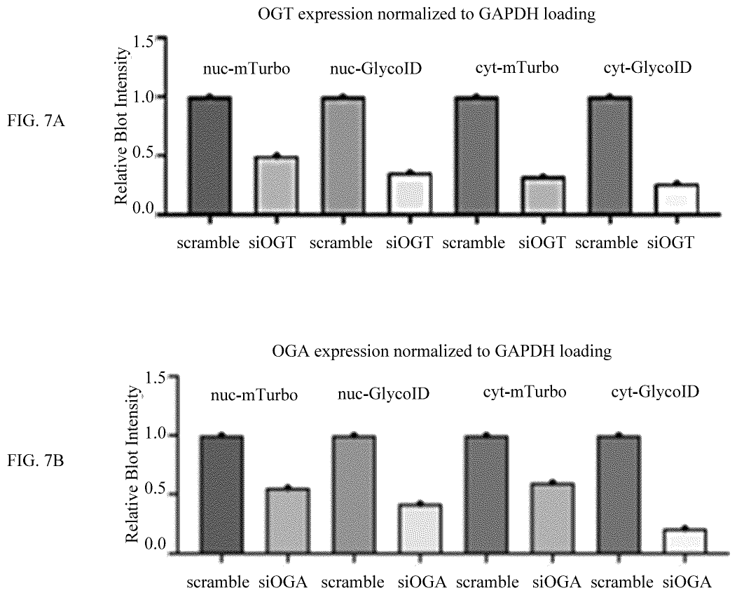

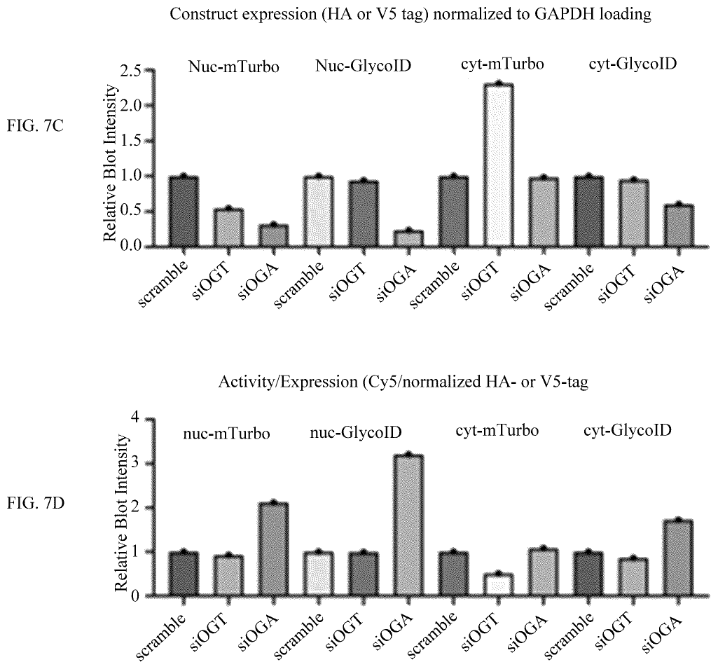

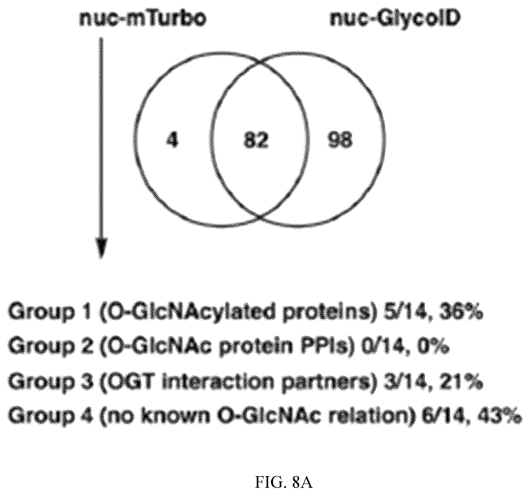

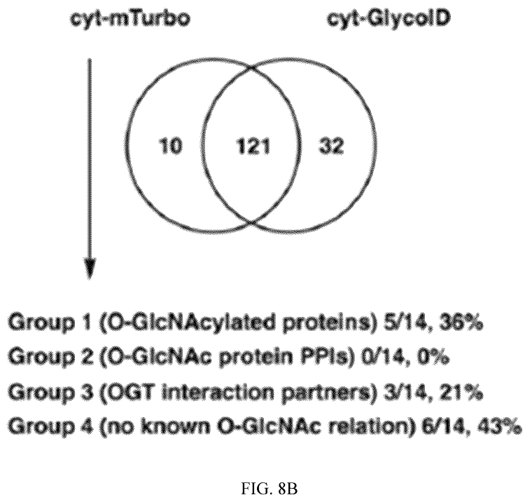

A noticeable effect with transient OGA knockdown was observed. Longer knockdown times or increased siRNA concentrations could increase the effect, but some cell toxicity during the OGT knockdown was observed. Tight regulation of OGT and OGA levels is well-documented and these short knockdown studies led to concomitant, feedback-based reduction of OGT and OGA that lessened the effects of these experiments. Complete blots for the GlcNAc engineering experiments were generated, alongside densitometry quantifications for additional clarity, see FIGS. 7A, 7B, 7C, and 7D.

Overall, the O-GlcNAc suppression and elevation results had a subtle effect on GlycoID-driven labeling patterns, with slightly increased labeling upon OGA knockdown but little to no impact upon OGT knockdown for 24 h. Mammalian cells are well established to rapidly attenuate OGT and OGA protein levels upon inhibition or knockdown of either species to maintain O-GlcNAc homeostasis, so it is hypothesized that OGT/OGA regulation over the 24 hours periods used for O-GlcNAc elevation or suppression led to reduced impact on overall GlycoID labeling patterns.

### GlycoID Identifies Physical Hubs of O-GlcNAcylated Protein Clusters in Subcellular Space

The targeted constructs cyt-GlycoID versus nuc-GlycoID gave a striking difference in band patterns due to labeling in two different subcellular locations. To compare specific proteins between the two cellular compartments, tandem mass spectrometry (MS/MS)-based proteomics (LC-MS/MS) was performed to identify which proteins were labeled by the targeted GlycoID constructs. HeLa cells that expressed nuc-GlycoID were compared with cells that expressed nuc-mTurbo as a nonsugar directed control in replicates of four per condition. Each construct was expressed in Hela cells grown in DMEM media, which lack biotin, to prevent premature labeling, 0 μM biotin). Biotin labeling was induced at 48 hours post-expression by adding 100 μM biotin and incubating for 6 hours at 37° C. to obtain maximum intracellular labeling. Proteomic identification hits were chosen using the default significance (p<0.05) and fold-enrichment (log₂>0.5) cutoffs in Perseus (version 1.6.2.1). Hits also had to satisfy the condition of being detected in at least three of the four replicates and have at least three unique peptide matches during MaxQuant processing. Label-free quantification was used as a relative difference between mTurbo-only constructs and the full GlycoID constructs. Western blots were performed for the expression of (a) cyt-mTurbo, (b) cyt-GlycoID, (c) nuc-mTurbo, and (d) nuc-GlycoID in Hela cells used in proteomics experiments. Labeling was conducted for 6 hours with 100 μM biotin. Blots were imaged using the iBright™ FL1500 instrument. Blots confirmed similar labeling efficiency between all replicates.

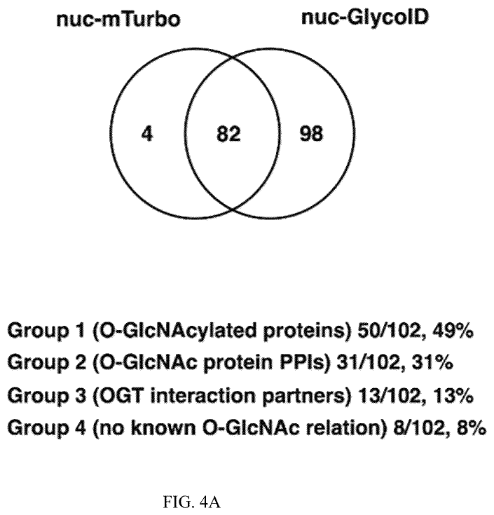

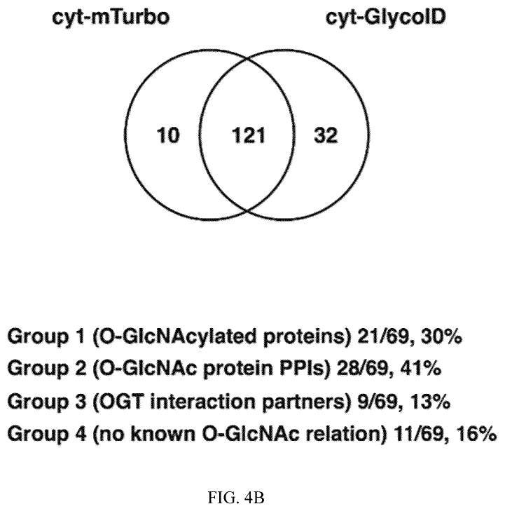

In the nuclear-targeted experiment, 98 proteins were identified exclusive to the nuc-GlycoID versus nuc-mTurbo condition, see FIG. 4A. Enrichment analysis between nuc-mTurbo and nuc-GlycoID showed statistically significant hits above the volcano plot. Volcano plot analysis showed an additional 4 proteins identified by nuc-GlycoID over nuc-mTurbo. Comparison with an O-GlcNAcome databank revealed that 49% of these 102 identified proteins were known O-GlcNAc proteins. A unique strength of proximity labeling is showing proteins that physically associate with the target proteins in subcellular space. Physical interactions between nuc-GlycoID hits revealed functional clusters with critical O-GlcNAc linkages with protein groups labeled by nuc-GlycoID.

To determine whether the remaining 51% of these proteins correspond to O-GlcNAc protein-binding partners, the STRING-db described in Szklarczyk D et al., Nucleic Acids Res. 2019, 47, D607-D613 was used to analyze reported protein-protein interactions (PPIs) between the nuc-GlycoID data set. The STRING settings only utilized experimentally verified PPIs. Interestingly, most non-O-GlcNAc protein hits (31/52) are known to have physical associations with O-GlcNAc proteins labeled by nuc-GlycoID. Based on the labeling radius of TurboID, described in Branon TC et al., Nat. Biotechnol 2018, 36, 880-887 these proteins were expected to be within 10 nm of the target-bound GlcNAc glycoproteins. Gene ontology analysis using k-means clustering revealed that these associated proteins comprised five functional O-GlcNAc hubs: mRNA binding, transcription factors, nucleotide binding, gene expression, and splicing. The PPI enrichment p-value, which compares the observed connections (edges between nodes) in the interaction network against what would be expected with no enrichment, averaged between p=1.19×10⁻⁵ and <1.0×10⁻¹⁶. For example, the highly connected splicing cluster had 60 observed PPI edges in the STRING analysis versus the expected number of five “random” PPIs based on the sizes of these proteins alone. This extremely high enrichment of functional protein activity hubs reveals that nuc-GlycoID labeled known interaction partners of O-GlcNAcylated proteins with very high confidence (significance). A summary of the clustering analysis with essential O-GlcNAc proteins identified is shown in Table 1.

TABLE 1
GlycoID Labels O-GlcNAc Clusters in Defined Cell Locations.
Full Activity Conditions: 6 hours with 100 μM Biotin

| | | | | PPI | |
|---|---|---|---|---|---|
| | | # of nodes | # of edges | enrichment | Sample |
| | Cluster | (proteins) | (interactions) | (p-value) | proteins |
| nuc- | mRNA binding | 28 | 46 | <1.0 × 10⁻¹⁶ | DHX9, RMB14, |
| GlycoID | | | | | ZFR, WBP11 |
| | Splicing | 19 | 60 | <1.0 × 10⁻¹⁶ | SF1, SF3A1, |
| | | | | | SF3B2, RBM25 |
| | Transcription | 19 | 5 | 4.59 × 10⁻⁵ | HCFC1, JunB, |
| | factors | | | | CCAR2, FUBP1 |
| | Gene | 19 | 14 | 1.19 × 10⁻⁵ | SYMPK, MBNL1, |
| | expression | | | | NONO, COIL, |
| | | | | | AGFG1 |
| | Metabolism | 14 | 7 | 5.41 × 10⁻⁵ | NOLC1, DHDH4, |
| | and signaling | | | | DIDO1, NKRF |
| Nuc- | None | 14 | 1 | 1.0 (no | HSPA8, NUMA, |
| mTurbo | | | | significance) | HIST1H1D |
| cyt- | Translation | 26 | 31 | 2.39 × 10⁻¹⁰ | EF1A1, RPL18, |
| GlycoID | | | | | LYAR, LLPH |
| | RNA binding | 24 | 10 | 3.34 × 10⁻⁶ | GNL3, RRP1B, |
| | | | | | THOC2 |
| | Cytoskeleton | 17 | 16 | 6.66 × 10⁻¹⁶ | ACTB, ACTA, |
| | | | | | RRBP1 |
| cyt- | Translation | 22 | 48 | 4.44 × 10⁻¹⁶ | RPL3, EIF5B, |
| mTurbo | | | | | RPS26 |
| | Glycolysis | 7 | 6 | 2.217 × 10⁻¹¹ | GAPDH, ALDOA, |
| | | | | | ENO1 |
| | Ubiquitinylation | 9 | 2 | 0.00226 | UBAP2, TRIP12 |
| | Spliceosome | 12 | 2 | 0.0337 | SRSF11, SREK1, |
| | | | | | U2SURP |

Further analysis of possible sources of O-GlcNAc-driven labeling on the remaining proteins without known O-GlcNAc sites or PPIs with O-GlcNAcylated proteins was performed using OGT-PIN, the O-GlcNAc transferase protein-interaction network described in Ma J et al., Int. J. Mol. Sci 2021, 22, 9620, OGT is known to form functional complexes with various activity hubs in cells, including histone chaperone complexes and tet protein DNA demethylation complexes, and there is a strong likelihood of coincidental proximity-based labeling of proteins within a 10 nm radius of an OGT hub. The OGT-PIN was used to reveal that a third group of proteins labeled by GlycoID constructs overlap with OGT-PIN data. The GlycoID analysis was divided into four groups: Group 1 proteins with known O-GlcNAc sites; Group 2 proteins that interact with O-GlcNAcylated proteins (via STRING-db); Group 3 proteins that form complex with OGT; and Group 4 proteins with no O-GlcNAc connection, likely experimental noise from high-abundance proteins such as thioredoxin. The nuc-GlycoID results from HeLa cells are summarized in FIG. 4D.

STRING analysis was performed on the nontargeted nuc-mTurbo-only constructs. The nuc-mTurbo construct identified only 14 proteins in total. These proteins did not cluster into any discrete functions, see Table 1, and were primarily high-abundance proteins such as heat shock proteins (HSPA8), histones (HIST1H1D), and microtubule-binding proteins (NUMA1), which indicated that nuclear-mTurboID labeling was dictated more by protein abundance.

A cytosolic GlycoID experiment was performed by comparing cyt-GlycoID labeling with cyt-mTurbo-expressing HeLa cells. After a 6 hours induction with 100 μM biotin, 32 proteins were exclusive to the cyt-GlycoID, see FIG. 4B. Volcano plot analysis revealed an additional 37 proteins significantly identified between cyt-GlycoID and cyt-mTurbo. STRING-db analysis of PPIs reveals that cyt-GlycoID can also identify functional O-GlcNAc hubs in the cytosol of cells. For cyt-GlycoID, 30% of the data set (21 hits) was known O-GlcNAc proteins, and the majority of the non-O-GlcNAc labeled hits (28 of the remaining 49) are known to be physically associated with these O-GlcNAc proteins. The cyt-GlycoID results in HeLa cells gave three significant clusters: RNA binding, cytoskeleton dynamics, and translation. The PPI enrichment p-values ranged from p=3.34×10⁻⁶ to 6.66×10⁻¹⁶, revealing that cyt-GlycoID labels known protein clusters with high functional significance. A summary of cyt-GlycoID clusters and important O-GlcNAc proteins is found in Table 1.

Conversely, four significant functional clusters were observed with cyt-mTurbo: The cyt-mTurbo functional clusters had diverging roles of ubiquitinylation, glycolysis, spliceosome (not observed in cyt-GlycoID), and one overlapping role, translation see Table 1. These different labeled functions indicated that cyt-mTurbo and cyt-GlycoID were directed to and labeled alternative complexes over the 6 hours labeling period.

Among the directly O-GlcNAcylated proteins observed with nuc-GlycoID, HCFC1, JunB, SF1, and ZFR stood out because they are among the top 10% of the O-GlcNAcome, based on the “O-GlcNAc score” from 0 to 100 that ranks the strength of the evidence for an O-GlcNAc site on a given protein. These nuclear proteins are most involved in transcriptional regulation and in production and splicing of mRNA, dynamic nuclear functions that O-GlcNAc is known to regulate. Among the cyt-GlycoID hits, EF1A1, ACTB, and RRBP1 have high O-GlcNAc scores and are involved in translation and cytoskeletal movements, two essential cytosolic functions regulated by O-GlcNAcylation. Several of the most common O-GlcNAcylated proteins, the nucleoporins were not seen, although their placement in the nuclear membrane might preclude GlycoID constructs from physically associating in the O-GlcNAcylated pore regions of these proteins.

One of the distinctive features of using an O-GlcNAc-targeted proximity labeling system according to aspects of the present disclosure is the ability to observe O-GlcNAcylated functional hubs made up of protein-protein interactions. The extremely high number of PPIs (up to 60) and the strong p-values between 10-5 and <10-16 demonstrate that the GlycoID strategy identified O-GlcNAcylated proteins and their physiological interaction partners. These O-GlcNAc interactomes may also be proximally involved in O-GlcNAc-regulated functions. These major clusters focus on transcription and mRNA splicing in the nucleus and translation in the cytosol, consistent with known O-GlcNAc transferase roles in mammalian cell proliferation and nutrient sensing.

Functional O-GlcNAc Glycoproteomics of Nutrient Sensing and Insulin Signaling

The intracellular nature of GlycoID allows monitoring of O-GlcNAc events in real-time and in localized subcellular space. A proteomic analysis workflow was used to analyze O-GlcNAc-related functions in cells to compare the effects of insulin stimulation following overnight serum starvation. Insulin is known to trigger changes in OGT and O-GlcNAcylation levels. Furthermore, engagement of the insulin receptor causes a rapid shift in OGT localization from the nucleus to the plasma membrane and cytosol between 5 and 30 min. After 60 min, OGT leaves the plasma membrane and returns to the nucleus. Therefore, the spatiotemporal features of the GlycoID compositions and methods according to aspects of the present disclosure track the functional effects of O-GlcNAc during insulin signaling.

It was hypothesized that nuc-GlycoID and cyt-GlycoID could detect critical changes in O-GlcNAc-driven functional hubs following starvation versus stimulation. For these experiments, the labeling time was reduced to 30 min to fall within the known time that insulin is known to trigger changes in OGT activity. HeLa cells were used for this experiment (ATCC #CCL-2), which display intact insulin receptor expression and signaling via Akt-Ser473 phosphorylation.

Western blots were performed for the expression and activity of cyt-mTurbo, cyt-GlycoID, nuc-mTurbo, and nuc-GlycoID in serum-starved Hela cells used in proteomics experiments. Labeling was conducted for 0.5 hours with 500 M biotin. Blots were imaged using the iBright™ FL1500 instrument.

The labeling activity of 1) cyt/nuc-GlycoID with HeLa cells supplemented with 5 μg/mL insulin with 0.5 hours of labeling with 500 μM biotin, 2) cyt/nuc-GlycoID with HeLa cells with 10% FBS with 0.5 hours of labeling and 500 μM biotin were determined. The effect of insulin on Akt blotted against Phospho-Akt (Ser473) antibody/Anti-Rabbit (1:10,000) was assayed and it was found that incubating the cells with insulin causes the phosphorylation of Akt.

The initial characterization produced reliable labeling at short time points at higher biotin concentration, the biotin in these experiments was raised to 500 μM for these 30 min labeling reactions. Four replicates of each condition were performed, and hits chosen were observed in at least three of four for the analysis for nuc-GlycoID starved, +serum, or +insulin and cyt-GlycoID starved, +serum, or +insulin. For the starved cyt-GlycoID version, 2/4 proteomic runs failed to give quality data sets. For this condition alone (cyt-GlycoID, starved), hits were chosen that were observed in both successful proteomic replicates. Statistical validation was performed in Perseus using the default cutoffs of p=0.05, fold-change >+0.5, and at least three unique peptide matches for a protein to be assigned as a high-confidence hit for further analysis.

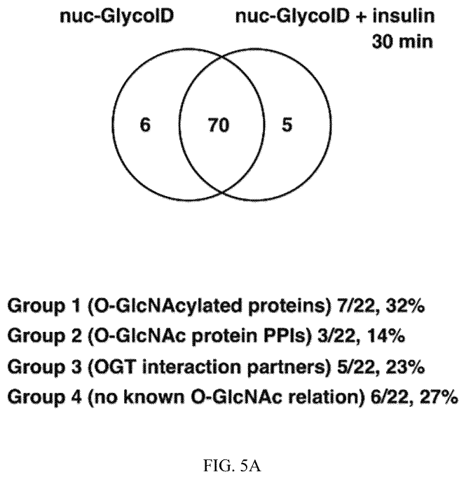

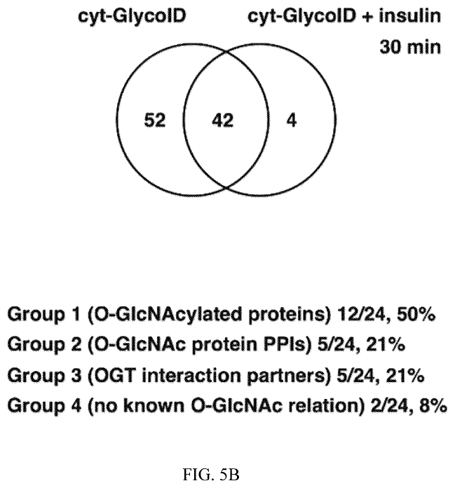

Complete protein blots confirmed labeling efficiency between all replicates. nuc-GlycoID was used to compare O-GlcNAc proteins between serum-starved and insulin-stimulated O-GlcNAc-related proteins in the nucleus. Changes were observed between the nucleus, where 19 proteins were differentially identified between the starved cells and 22 were identified between the insulin-stimulated cells, see FIG. 5A. Of the identified proteins in the nuc-GlycoID insulin data set, a relatively low 32% was O-GlcNAc-modified. STRING analysis was used to identify reported protein interactions between the hits. The OGT-PIN set was used to analyze the OGT interactors and split the analysis into the O-GlcNAc-related Groups 1-4, as detailed above. A k-means clustering analysis in STRING revealed only one significant functional group involved in the preribosomal assembly. The remaining proteins did not fall into a substantial grouping but included O-GlcNAcylated regulatory proteins such as ABCF1 (translation initiation) and CDK12, a kinase involved in regulating the cell cycle.⁵⁰ The k-means clustering results are summarized in Table 2.

TABLE 2
Functional O-GlcNAc Labeling in Stimulated vs Serum-Starved Cells^{a}

| | | | | PPI | |
|---|---|---|---|---|---|
| | | # of nodes | # of edges | enrichment | Sample |
| | Cluster | (proteins) | (interactions) | (p-value) | proteins |
| nuc- | Pre-ribosome | 8 | 2 | 2.84 × 10⁻³ | GLN3, RPS8, |
| GlycoID + | assembly | | | | PRS26 |
| insulin | | | | | |
| | No cluster | 22 | 5 | 0.0548 (not | CDK12 |
| | | | | significant) | (signaling), |
| | | | | | ABCF1 |
| | | | | | (translation) |
| cyt- | Translation | 11 | 9 | 7.30 × 10⁻⁴ | RRP1B, EIF5B, |
| GlycoID + | | | | | RPL29 |
| insulin | | | | | |
| | RNA binding | 8 | 1 | 0.041 | RBBP6, SRSF11, |
| | | | | | MKI67 |
| nuc- | RNA binding | 9 | 4 | 2.83 × 10⁻⁶ | LYAR, GL3, |
| GlycoID + | | | | | LLPH |
| serum (10%) | | | | | |
| | Pre-splicosome | 6 | 2 | 4.56 × 10⁻⁴ | KNOP1, RBM39 |
| | Pre-ribosome | 4 | 1 | 0.0409 | BRIX1, DDX24 |
| cyt- | Translation | 14 | 17 | 1.05 × 10⁻¹ | LYAR, ABCF1, |
| GlycoID + | | | | | EIF5B |
| serum (10%) | | | | | |
| | Pre-ribosome | 10 | 5 | 4.27 × 10⁻⁵ | RBM39, RRBP1, |
| | | | | | RRP1B |

^{a}Labeling: 30 min with 500 μM biotin

The cytosolic analysis revealed 65 starved hits versus 24 insulin-driven proteins, see FIG. 5B. Enrichment analysis between starved and insulin conditions was performed producing a volcano plot with statistically significant hits. Physical interactions between nuc-GlycoID/insulin hits revealed one functional cluster with key O-GlcNAc linkages. NS=not significant. The protein groups labeled by nuc-GlycoID/insulin. STRING analysis and k-means clustering identified two significant clusters, primarily involved in translation and RNA binding. There were also several nonclustered proteins involved in actin dynamics (FNLA) and fatty acid biogenesis (ACACA), two metabolic features that respond to insulin. The Group 1-4 analysis revealed that, for known O-GlcNAc proteins, cyt-GlycoID (50% Group 1 labeling) worked moderately better than nuc-GlycoID (32% Group 1 labeling) under insulin, 30 min stimulation. The functional k-means clustering results are summarized in Table 2.

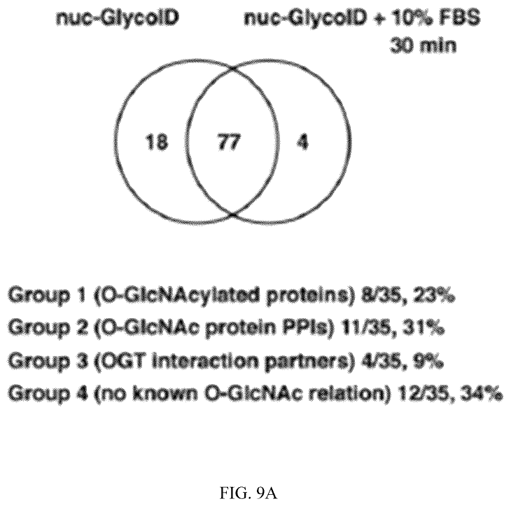

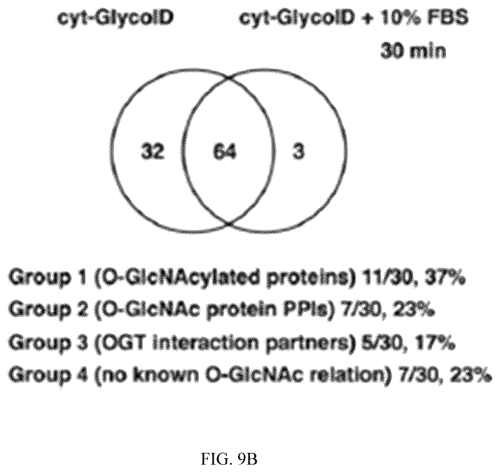

Serum-starved cells were used to compare with serum-fed conditions, again at the 30 min time point with 500 μM biotin. Because serum contains both nutrients and growth factors, it was hypothesized that GlycoID labeling patterns from cells stimulated by serum would also display different functional hubs. Proteomic analysis was conducted for the insulin labeling, see FIGS. 9A, 9B. Three significant clusters were observed in the nuc-GlycoID+serum data set, RNA binding, prespliceosome assembly, and preribosome assembly. Two significant clusters were observed in the cyt-GlycoID+serum data set: translation and preribosome assembly.

Overall, fewer total proteins were observed in both functional experiments compared to the first analysis of mTurbo versus GlycoID constructs. This observation of fewer proteins identified may come from two potential reasons: first, the labeling time was 0.5 versus 6 hours (albeit at higher biotin concentration); second, in the nature of the comparison. Second, GlycoID was compared with or without insulin and serum in these functional experiments. Therefore, any overlapping O-GlcNAcylated proteins that did not change in O-GlcNAc status during stimulation during GlycoID labeling between starved and stimulated would not be observed. This overlap is expected due to the widespread distribution of O-GlcNAc on proteins; not all proteins will change O-GlcNAc following growth factor stimulation. These labeling results suggested that only a subset of O-GlcNAcylated proteins actively responded to insulin or serum stimulation at the 30 min time point, which is functionally interesting.

Longer induction times might reveal more widespread changes in O-GleNAc patterns and interactomes. In the insulin labeling experiment, diminished nuc-GlycoID labeling and enhanced cyt-GlycoID labeling was observed, which is approximately the inverse of what was observed under steady-state cell conditions, where OGT is more active in the nucleus (compare FIG. 4B versus FIG. 5B). This spatiotemporal effect might reflect the movement of OGT toward the plasma membrane that is reported to peak at 30 min, therefore losing some activity in the nucleus and cytosol. Backing up this hypothesis, some of the canonical O-GlcNAcylated insulin-signaling proteins that were expected were not observed, including Akt, PDK1, and IRS1, which may reflect their membrane-associated nature during insulin stimulation.

The results presented herein show tracking O-GlcNAc dynamics in live cells over a variety of homeostasis, signaling, and pathological conditions.

These results show that spatial targeting of fusion proteins according aspects of the present disclosure including cellular localization signals revealed different labeling patterns between O-GlcNAc interactomes in the nucleus versus cytosol. Furthermore, functional O-GlcNAc labeling experiments conducted for short, 30 min periods during insulin or serum stimulation demonstrated the ability to track O-GlcNAcylation patterns and interactome changes in real-time. This functional O-GlcNAc interactome data adds evidence to a growing area of OGT-regulated hubs of activity, including splicing, metabolism, and signaling.

### REFERENCES

(1). Hart GW; Housley MP; Slawson C Cycling of O-linked B-N-acetylglucosamine on nucleocytoplasmic proteins. Nature 2007, 446, 1017-1022. [PubMed: 17460662]
(2). Chatham J; Marchase R Protein O-GlcNAcylation: A critical regulator of the cellular response to stress. Curr. Signal Transduction Ther 2010, 5, 49-59.
(3). Lund PJ; Elias JE; Davis MM Global Analysis of O-GlcNAc Glycoproteins in Activated Human T Cells. J. Immunol 2016, 197, 3086-3098. [PubMed: 27655845]
(4). Woo CM; Lund PJ; Huang AC; Davis MM; Bertozzi CR; Pitteri SJ Mapping and quantification of over 2000 O-linked glycopeptides in activated human T cells with isotope-targeted glycoproteomics (Isotag). Mol. Cell. Proteomics 2018, 17, 764-775. [PubMed: 29351928]
(5). Nagy T; Fisi V; Frank D; Kátai E; Nagy Z; Miseta A Hyperglycemia-Induced Aberrant Cell Proliferation; A Metabolic Challenge Mediated by Protein O-GlcNAc Modification. Cells 2019, 8, 999.
(6). Masaki N; Feng B; Bretón-Romero R; Inagaki E; Weisbrod RM; Fetterman JL; Hamburg NM O-GlcNAcylation Mediates Glucose-Induced Alterations in Endothelial Cell Phenotype in Human Diabetes Mellitus. J. Am. Heart Assoc 2020, 9, No. e014046. [PubMed: 32508185]
(7). Dupas T; Denis M; Dontaine J; Persello A; Bultot L; Erraud A; Vertommen D; Bouchard B; Dhot J; De Waard M; Olson A; Rozec B; Rosiers CD; Bertrand L; Issad T; Lauzier B O-GlcNAc levels are regulated in a tissue and time specific manner during post-natal development, independently of dietary intake. Arch. Cardiovasc. Dis. Suppl 2020, 12, 221.
(8). Zachara NE; Molina H; Wong KY; Pandey A; Hart GW The dynamic stress-induced “O-GlcNAc-ome” highlights functions for O-GlcNAc in regulating DNA damage/repair and other cellular pathways. Amino Acids 2011, 40, 793-808. [PubMed: 20676906]
(9). Levine ZG; Walker S The Biochemistry of O-GlcNAc Transferase: Which Functions Make It Essential in Mammalian Cells? Annu. Rev. Biochem 2016, 85, 631-657. [PubMed: 27294441]
(10). Ju Kim E O-GlcNAc Transferase: Structural Characteristics, Catalytic Mechanism and Small-Molecule Inhibitors. ChemBioChem 2020, 21, 3026-3035. [PubMed: 32406185]
(11). Yang X; Ongusaha PP; Miles PD; Havstad JC; Zhang F; So WV; Kudlow JE; Michell RH; Olefsky JM; Field SJ; Evans RM Phosphoinositide signalling links O-GlcNAc transferase to insulin resistance. Nature 2008, 451, 964-969. [PubMed: 18288188]
(12). Taylor RP; Geisler TS; Chambers JH; McClain DA Up-regulation of O-GlcNAc Transferase with Glucose Deprivation in HepG2 Cells Is Mediated by Decreased Hexosamine Pathway Flux. J. Biol. Chem 2009, 284, 3425-3432. [PubMed: 19073609]
(13). Zhang Z; Tan EP; VandenHull NJ; Peterson KR; Slawson C O-GlcNAcase Expression is Sensitive to Changes in O-GlcNAc Homeostasis. Front. Endocrinol 2014, 5, 206.
(14). Tan Z-W; Fei G; Paulo JA; Bellaousov S; Martin SES; Duveau DY; Thomas CJ; Gygi SP; Boutz PL; Walker S O-GlcNAc regulates gene expression by controlling detained intron splicing. Nucleic Acids Res. 2020, 48, 5656-5669. [PubMed: 32329777]
(15). Marshall S; Nadeau O; Yamasaki K Dynamic Actions of Glucose and Glucosamine on Hexosamine Biosynthesis in Isolated Adipocytes: Differential Effects on Glucosaomine 6-Phosphate, UDP—N-Acetylglucosamine, and ATP Levels. J. Biol. Chem 2004, 279, 35313-35319. [PubMed: 15199059]
(16). Champattanachai V; Marchase RB; Chatham JC Glucosamine protects neonatal cardiomyocytes from ischemia-reperfusion injury via increased protein-associated O-GlcNAc. Am. J. Physiol.: Cell Physiol 2007, 292, C₁₇₈-C₁₈₇. [PubMed: 16899550]
(17). Song M; Kim H-S; Park J-M; Kim S-H; Kim I-H; Ryu SH; Suh P-G O-GlcNAc transferase is activated by CaMKIV-dependent phosphorylation under potassium chloride-induced depolarization in NG-108-15 cells. Cell. Signalling 2008, 20, 94-104. [PubMed: 18029144]
(18). Dias WB; Cheung WD; Wang Z; Hart GW Regulation of Calcium/Calmodulin-dependent Kinase IV by O-GlcNAc Modification. J. Biol. Chem 2009, 284, 21327-21337. [PubMed: 19506079]
(19). Joeh E; O'Leary T; Li W; Hawkins R; Hung JR; Parker CG; Huang ML Mapping glycan-mediated galectin-3 interactions by live cell proximity labeling. Proc. Natl. Acad. Sci. U.S.A 2020, 117, 27329-27338. [PubMed: 33067390]
(20). Groves JA; Maduka AO; O'Meally RN; Cole RN; Zachara NE Fatty acid synthase inhibits the O-GlcNAcase during oxidative stress. J. Biol. Chem 2017, 292, 6493-6511. [PubMed: 28232487]
(21). Stephen HM; Praissman JL; Wells L Generation of an Interactome for the Tetratricopeptide Repeat Domain of O-GlcNAc Transferase Indicates a Role for the Enzyme in Intellectual Disability. J. Proteome Res 2021, 20, 1229-1242. [PubMed: 33356293]
(22). Yang X; Qian K Protein O-GlcNAcylation: emerging mechanisms and functions. Nat. Rev. Mol. Cell Biol 2017, 18, 452. [PubMed: 28488703]
(23). Gorelik A; van Aalten DMF Tools for functional dissection of site-specific O-GlcNAcylation. RSC Chem. Biol 2020, 1, 98-109. [PubMed: 34458751]
(24). Branon TC; Bosch JA; Sanchez AD; Udeshi ND; Svinkina T; Carr SA; Feldman JL; Perrimon N; Ting AY Efficient proximity labeling in living cells and organisms with TurboID. Nat. Biotechnol 2018, 36, 880-887. [PubMed: 30125270]
(25). Saarela S; Westerlund-Wikström B; Rhen M; Korhonen TK The GafD protein of the G (F17) fimbrial complex confers adhesiveness of Escherichia coli to laminin. Infect. Immun 1996, 64, 2857-2860. [PubMed: 8698525]
(26). Carrillo LD; Froemming JA; Mahal LK Targeted in vivo O-GlcNAc sensors reveal discrete compartment-specific dynamics during signal transduction. J. Biol. Chem 2011, 286, 6650-6658. [PubMed: 21138847]
(27). Saarela S; Taira S; Nurmiaho-Lassila EL; Makkonen A; Rhen M The Escherichia coli G-fimbrial lectin protein participates both in fimbrial biogenesis and in recognition of the receptor N-acetyl-D-glucosamine. J. Bacteriol 1995, 177, 1477-1484. [PubMed: 7883703]
(28). Xu Y; Beckett D Kinetics of biotinyl-5′-adenylate synthesis catalyzed by the Escherichia coli repressor of biotin biosynthesis and the stability of the enzyme-product complex. Biochemistry 1994, 33, 7354-7360. [PubMed: 8003500]
(29). Hsu K-L; Gildersleeve JC; Mahal LK A simple strategy for the creation of a recombinant lectin microarray. Mol. BioSyst 2008, 4, 654-662. [PubMed: 18493664]
(30). Carrillo LD; Krishnamoorthy L; Mahal LK A Cellular FRET-Based Sensor for B-O-GlcNAc, A Dynamic Carbohydrate Modification Involved in Signaling. J. Am. Chem. Soc 2006, 128, 14768-14769. [PubMed: 17105262]
(31). Merckel MC; Tanskanen J; Edelman S; Westerlund-Wikström B; Korhonen TK; Goldman A The structural basis of receptor-binding by Escherichia coli associated with diarrhea and septicemia. J. Mol. Biol 2003, 331, 897-905. [PubMed: 12909017]
(32). Duckworth BP; Geders TW; Tiwari D; Boshoff HI; Sibbald PA; Barry CE; Schnappinger D; Finzel BC; Aldrich CC Bisubstrate Adenylation Inhibitors of Biotin Protein Ligase from Mycobacterium tuberculosis. Chem. Biol 2011, 18, 1432-1441. [PubMed: 22118677]
(33). Wulff-Fuentes E; Berendt RR; Massman L; Danner L; Malard F; Vora J; Kahsay R; Olivier-Van Stichelen S The human O-GlcNAcome database and meta-analysis. Sci. Data 2021, 8, 25. [PubMed: 33479245]
(34). Olivier-Van Stichelen S; Wang P; Comly M; Love DC; Hanover JA Nutrient-driven O-linked N-acetylglucosamine (O-GlcNAc) cycling impacts neurodevelopmental timing and metabolism. J. Biol. Chem 2017, 292, 6076-6085. [PubMed: 28246173] (35). Nagy T; Fisi V; Frank D; Kátai E; Nagy Z; Miseta A Hyperglycemia-Induced Aberrant Cell Proliferation; A Metabolic Challenge Mediated by Protein O-GlcNAc Modification. Cells 2019, 8, 999.
(36). Baumann D; Wong A; Akhaphong B; Jo S; Pritchard S; Mohan R; Chung G; Zhang Y; Alejandro EU Role of nutrient-driven O-GlcNAc-post-translational modification in pancreatic exocrine and endocrine islet development. Development 2020, 147, dev186643. [PubMed: 32165492]
(37). Park S-K; Zhou X; Pendleton KE; Hunter OV; Kohler JJ; O'Donnell KA; Conrad NK A Conserved Splicing Silencer Dynamically Regulates O-GlcNAc Transferase Intron Retention and O-GlcNAc Homeostasis. Cell Rep. 2017, 20, 1088-1099. [PubMed: 28768194]
(38). Samavarchi-Tehrani P; Samson R; Gingras A-C Proximity Dependent Biotinylation: Key Enzymes and Adaptation to Proteomics Approaches. Mol. Cell. Proteomics 2020, 19, 757-773. [PubMed: 32127388]
(39). Szklarczyk D; Gable AL; Lyon D; Junge A; Wyder S; Huerta-Cepas J; Simonovic M; Doncheva NT; Morris JH; Bork P; Jensen LJ; Mering CV STRING v11: protein-protein association networks with increased coverage, supporting functional discovery in genome-wide experimental datasets. Nucleic Acids Res. 2019, 47, D607-d613. [PubMed: 30476243]
(40). Ma J; Hou C; Li Y; Chen S; Wu C OGT Protein Interaction Network (OGT-PIN): A Curated Database of Experimentally Identified Interaction Proteins of OGT. Int. J. Mol. Sci 2021, 22, 9620. [PubMed: 34502531]
(41). Levine ZG; Potter SC; Joiner CM; Fei GQ; Nabet B; Sonnett M; Zachara NE; Gray NS; Paulo JA; Walker S Mammalian cell proliferation requires noncatalytic functions of O-GlcNAc transferase. Proc. Natl. Acad. Sci. U.S.A 2021, 118, No. e2016778118. [PubMed: 33419956]
(42). Lee J-S; Zhang Z O-linked N-acetylglucosamine transferase (OGT) interacts with the histone chaperone HIRA complex and regulates nucleosome assembly and cellular senescence. Proc. Natl. Acad. Sci. U.S.A 2016, 113, E3213-E3220. [PubMed: 27217568]
(43). Vella P; Scelfo A; Jammula S; Chiacchiera F; Williams K; Cuomo A; Roberto A; Christensen J; Bonaldi T; Helin K; Pasini D Tet Proteins Connect the O-Linked N-acetylglucosamine

Transferase Ogt to Chromatin in Embryonic Stem Cells. Mol. Cell 2013, 49, 645-656. [PubMed: 23352454]
(44). Ozcan S; Andrali SS; Cantrell JE Modulation of transcription factor function by O-GlcNAc modification. Biochim. Biophys. Acta 2010, 1799, 353-364. [PubMed: 20202486]
(45). Tarbet HJ; Dolat L; Smith TJ; Condon BM; O'Brien ET III; Valdivia RH; Boyce M Site-specific glycosylation regulates the form and function of the intermediate filament cytoskeleton. eLife 2018 7, No. e31807. [PubMed: 29513221]
(46). Li X; Zhu Q; Shi X; Cheng Y; Li X; Xu H; Duan X; Hsieh-Wilson LC; Chu J; Pelletier J;

Ni M; Zheng Z; Li S; Yi W O-GlcNAcylation of core components of the translation initiation machinery regulates protein synthesis. Proc. Natl. Acad. Sci. U.S.A 2018 116, 7857-7866.
(47). Perez-Cervera Y; Dehennaut V; Aquino Gil M; Guedri K; Solórzano Mata CJ; Olivier-Van Stichelen S; Michalski JC; Foulquier F; Lefebvre T Insulin signaling controls the expression of O-GlcNAc transferase and its interaction with lipid microdomains. FASEB J. 2013, 27, 3478-3486. [PubMed: 23689613]
(48). Majumdar G; Wright J; Markowitz P; Martinez-Hernandez A; Raghow R; Solomon SS Insulin Stimulates and Diabetes Inhibits O-Linked N-Acetylglucosamine Transferase and O-Glycosylation of Sp1. Diabetes 2004, 53, 3184-3192. [PubMed: 15561949]
(49). Nagarajan A; Petersen MC; Nasiri AR; Butrico G; Fung A; Ruan H-B; Kursawe R; Caprio S; Thibodeau J; Bourgeois-Daigneault M-C; Sun L; Gao G; Bhanot S; Jurczak MJ; Green MR; Shulman GI; Wajapeyee N MARCH1 regulates insulin sensitivity by controlling cell surface insulin receptor levels. Nat. Commun 2016, 7, 12639. [PubMed: 27577745]
(50). Schwein PA; Woo CM The O-GlcNAc Modification on Kinases. ACS Chem. Biol 2020, 15, 602-617. [PubMed: 32155042]
(51). Slawson C; Copeland RJ; Hart GW O-GlcNAc signaling: a metabolic link between diabetes and cancer? Trends Biochem. Sci 2010, 35, 547-555. [PubMed: 20466550]

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A fusion protein comprising: a glycan binding component linked to a mutant E. coli biotin ligase BirA, the glycan binding component capable of specific binding to a glycosylation post-translational modification of a target protein and the mutant E. coli biotin ligase BirA having enzymatic activity to ligate biotin to proteins proximal to the target protein.

2. The fusion protein of claim 1, wherein the glycan binding component is selected from the group consisting of: a lectin, a collectin, a ficolin, a C-reactive protein, and a carbohydrate-binding domain of any thereof.

3. The fusion protein of claim 1, wherein the glycan binding component is selected from the group consisting of: an aptamer, an antibody, and an antigen-binding fragment of an antibody.

4. The fusion protein of claim 1, wherein the glycan binding component is GafD lectin.

5. The fusion protein of claim 1, wherein the mutant E. coli biotin ligase BirA comprises SEQ ID NO:1, SEQ ID NO:2, or a variant of either thereof having enzymatic activity to ligate biotin to proteins proximal to the target protein.

6. The fusion protein of claim 1, wherein the glycan binding component has a C-terminus and an N-terminus, the mutant E. coli biotin ligase BirA has a C-terminus and an N-terminus, and the C-terminus of the glycan binding component is linked to the N-terminus of the mutant E. coli biotin ligase BirA.

7. The fusion protein of claim 1, wherein the glycan binding component is linked to the mutant E. coli biotin ligase BirA by a linker disposed between the glycan binding component and the mutant E. coli biotin ligase BirA.

8. The fusion protein of claim 1, further comprising a localization signal peptide.

9. The fusion protein of claim 1, further comprising a localization signal peptide capable of promoting localization of the fusion protein to a subcellular compartment selected from the group consisting of: nucleus, cytosol, mitochondria, endoplasmic reticulum, and plasma membrane.

10. The fusion protein of claim 1, further comprising an exogenous detectable tag.

11. A method of detecting proteins proximal to a target protein, comprising:
contacting a living cell with the fusion protein according to claim 1 under compatible biological conditions, whereby the fusion protein specifically binds to a glycosylation post-translational modification of a target protein of the cell;
providing biotin to the living cell, whereby the mutant E. coli biotin ligase BirA ligates biotin to proteins proximal to the target protein; and
detecting the biotinylated proteins, thereby detecting proteins proximal to the target protein.

12. The method of claim 11, wherein detecting the biotinylated proteins comprises purifying the biotinylated proteins and detecting the purified biotinylated proteins.

13. The method of claim 12, wherein detecting the purified biotinylated proteins comprises mass spectrometry.

14. The method of claim 12, wherein detecting the purified biotinylated proteins comprises chromatography.

15. The method of claim 14, wherein the chromatography comprises gel electrophoresis.

16. The method of claim 15, wherein the chromatography comprises gel electrophoresis and transfer of the electrophoresed purified biotinylated proteins to a membrane.

17. The method according to claim 11, wherein contacting the living cell with the fusion protein comprises introducing an expression construct encoding the fusion protein into the cell.

18. An expression construct comprising a nucleic acid encoding the fusion protein according to claim 1.

19. A cell comprising the expression construct of claim 18.
